(19) United States
(12) Patent Application Publication
(10) Pub. No.: US 2025/0059237 A1
(43) Pub. Date: Feb. 20, 2025

(54) METHODS OF FORMING RADIOPAQUE PEPTIDES AND MEDICAL HYDROGELS FORMED THEREFROM

(71) Applicant: Boston Scientific Scimed, Inc., Maple Grove, MN (US)

(72) Inventors: Joseph Thomas Delaney, JR., Minneapolis, MN (US); Rick Dorn, Plymouth, MN (US); Yen-Hao Hsu, Shrewsbury, MA (US); Cristian Parisi, Boston, MA (US)

(73) Assignee: Boston Scientific Scimed, Inc., Maple Grove, MN (US)

(21) Appl. No.: 18/804,679

(22) Filed: Aug. 14, 2024

Related U.S. Application Data
(60) Provisional application No. 63/519,962, filed on Aug. 16, 2023.
(60) Provisional application No. 63/618,654, filed on Jan. 8, 2024.

(51) Int. Cl. C07K 14/00; C07K 1/06; C07K 1/10; C07K 1/107

(52) U.S. Cl. CPC C07K 14/001; C07K 1/061; C07K 1/10; C07K 1/107

(57) ABSTRACT

In various aspects, the present disclosure pertains to methods of forming an iodinated peptide compound that comprises: (a) forming a multifunctional precursor compound comprising (i) a residue of an amine-protected peptide that comprises two or more amino acid residues with primary-amine-containing side groups and (ii) a multifunctional moiety linked to the amine-protected peptide residue through an amide group or an ester group; (b) reacting the multifunctional precursor compound in an amide coupling reaction or an esterification coupling reaction with an iodinated compound to form an iodinated, amine-protected peptide compound comprising n iodinated moieties that are covalently attached to the amine-protected peptide residue through a residue of the multifunctional moiety; and (c) deprotecting the iodinated, amine-protected peptide compound to form the iodinated peptide compound.

### CROSS REFERENCE TO RELATED APPLICATIONS

This application claims the benefit of U.S. Provisional Patent Application Ser. No. 63/519,962 filed on Aug. 16, 2023, and U.S. Provisional Patent Application Ser. No. 63/618,654 filed on Jan. 8, 2024, the disclosures of which are incorporated herein by reference.

### FIELD

The present disclosure relates to methods of forming radiopaque peptides, to the use of such radiopaque peptides as crosslinking agents for forming hydrogels, and to hydrogels formed from such radiopaque peptides. The radiopaque peptides and hydrogels are useful, for example, in various medical applications.

### BACKGROUND

SpaceOAR®, a rapid crosslinking hydrogel that polymerizes in vivo within seconds, is based on a multi-arm polyethylene glycol (PEG) polymer functionalized with succinimidyl glutarate as activated end groups which further react with trilysine to form crosslinks. This product has become a very successful, clinically-used biomaterial in prostate cancer therapy. A further improvement based on this structure is that a portion the succinimidyl glutarate end groups have been functionalized with 2,3,5-triiiodobenzamide groups, providing radiopacity. This hydrogel, known by the trade name of SpaceOAR Vue®, is the radiopaque version of SpaceOAR® for prostate medical applications.

Alternative strategies for forming iodine-labelled crosslinked hydrogels that provide enhanced radiopacity while maintaining crosslink density per polymer molecule are of interest for medical applications.

### SUMMARY

In various aspects, the present disclosure pertains to methods of forming an iodinated peptide compound that comprises: (a) forming a multifunctional precursor compound comprising (i) a residue of an amine-protected peptide that comprises two or more amino acid residues with primary-amine-containing side groups and (ii) a multifunctional moiety linked to the amine-protected peptide residue through an amide group or an ester group, the multifunctional moiety further comprising n primary amine groups, n hydroxyl groups, or n carboxyl groups, wherein n is an integer of 2 or more; (b) reacting the multifunctional precursor compound in an amide coupling reaction or an esterification coupling reaction with an iodinated compound selected from an amine-functional iodinated compound that comprises an iodinated moiety, a carboxyl-functional iodinated compound that comprises an iodinated moiety, and a hydroxy-functional iodinated compound that comprises an iodinated moiety, to form an iodinated, amine-protected peptide compound comprising n iodinated moieties that are covalently attached to the amine-protected peptide residue through a residue of the multifunctional moiety; and (c) deprotecting the iodinated, amine-protected peptide compound to form the iodinated peptide compound.

In some embodiments, which can be used in conjunction with the above aspects and embodiments, the multifunctional moiety is linked to the amine-protected peptide residue through the amide group and the multifunctional moiety comprises the n primary amine groups.

In some of these embodiments, the multifunctional precursor compound is formed by a process that comprises reacting in an amide coupling reaction (i) a polyamine compound having n+1 primary amine groups with (ii) a carboxyl group at a C-terminus of the amine-protected peptide that comprises the two or more amino acid residues with the primary-amine-containing side groups, the primary amine groups of the amine-protected peptide being protected from participation in the amide coupling reaction.

In some of these embodiments, the n primary amine groups of the multifunctional precursor compound are reacted in an amide coupling reaction with n carboxyl-functional iodinated compounds to form the iodinated, amine-protected peptide compound.

In some of these embodiments, the multifunctional moiety is a residue of a polyamine compound having n+1 primary amine groups, one of the n+1 primary amine groups being used to form the amide group that links the multifunctional moiety to the amine-protected peptide residue, and n of the n+1 primary amine groups being used in the amide coupling reaction between the multifunctional precursor compound and the carboxyl-functional iodinated compound that comprises the iodinated moiety.

In some embodiments, which can be used in conjunction with the above aspects and embodiments, the multifunctional moiety is linked to the amine-protected peptide residue through the ester group and the multifunctional moiety comprises the n hydroxyl groups.

In some of these embodiments, the multifunctional precursor compound is formed by a process that comprises reacting in an ester coupling reaction (i) a polyol compound having n+1 hydroxyl groups with (ii) a carboxyl group at a C-terminus of the amine-protected peptide that comprises the two or more amino acid residues with the primary-amine-containing side groups, the primary amine groups of the amine-protected peptide being protected from participation in the ester coupling reaction.

In some of these embodiments, the n hydroxyl groups of the multifunctional precursor compound are reacted in an ester coupling reaction with n of the carboxyl-functional iodinated compounds to form the iodinated, amine-protected peptide compound.

In some of these embodiments, the multifunctional moiety is a residue of a polyol compound having n+1 hydroxyl groups, one of the n+1 hydroxyl groups being to form the ester group that links the multifunctional moiety to the amine-protected peptide residue, and n of the n+1 hydroxyl groups being used in the ester coupling reaction between the multifunctional precursor compound and the carboxyl-functional iodinated compound that comprises the iodinated moiety.

In some embodiments, which can be used in conjunction with the above aspects and embodiments, the multifunctional moiety is linked to the amine-protected peptide residue through the amide group and the multifunctional moiety comprises the n carboxyl groups.

In some of these embodiments, the n carboxyl groups of the multifunctional precursor compound are reacted with n of the hydroxyl-functional iodinated compounds in an ester coupling reaction to form the iodinated, amine-protected peptide compound.

In some of these embodiments, the n carboxyl groups of the multifunctional precursor compound are reacted with n amine-functional iodinated compounds in an amide coupling reaction to form the iodinated, amine-protected peptide compound.

In some embodiments, which can be used in conjunction with the above aspects and embodiments, the multifunctional precursor compound is formed by a process that comprises (a) reacting in an amide coupling reaction (i) a polyamine compound having n+1 primary amine groups with (ii) a carboxyl group at a C-terminus of the amine-protected peptide that comprises the two or more amino acid residues with the primary-amine-containing side group, the primary amine groups of the amine-protected peptide being protected from participation in the amide coupling reaction, the reaction product having n primary amine groups; and (b) reacting the n primary amine groups of the reaction product of step (a) with a cyclic anhydride in a ring opening reaction, thereby forming the n carboxyl groups at the sites of the n primary amine groups.

In some embodiments, which can be used in conjunction with the above aspects and embodiments, the multifunctional moiety comprises a residue of a polyamine compound having n+1 primary amine groups, one of the n+1 primary amine groups being used to form the amide group that links the multifunctional moiety to the amine-protected peptide residue, n of the n+1 primary amine groups being used in a ring opening reaction with a cyclic anhydride to form the multifunctional moiety that comprises the n carboxyl groups, and the n carboxyl groups being used in the ester coupling reaction between the multifunctional precursor compound and the hydroxy-functional iodinated compound that comprises the iodinated moiety.

In some embodiments, which can be used in conjunction with the above aspects and embodiments, the multifunctional moiety comprises a residue of a polyamine compound having n+1 primary amine groups, one of the n+1 primary amine groups being used to form the amide group that links the multifunctional moiety to the amine-protected peptide residue, n of the n+1 primary amine groups being used in a ring opening reaction with a cyclic anhydride to form the multifunctional moiety that comprises the n carboxyl groups, and the n carboxyl groups being used in the amide coupling reaction between the multifunctional precursor compound and the amine-functional iodinated compound that comprises the iodinated moiety.

In some embodiments, which can be used in conjunction with the above aspects and embodiments, the multifunctional precursor compound is formed by a process that comprises (a) reacting in an amide coupling reaction (i) a carboxyl-protected polycarboxylamine compound comprising an amine group and n protected carboxyl groups that are protected from reacting in the amide coupling reaction with (ii) a carboxyl group at a C-terminus of an amine-protected peptide that comprises the two or more amino acid residues with the primary-amine-containing side groups, the primary amine groups of the amine-protected peptide being protected from reacting in the amide coupling reaction; and (b) deprotecting the n protected carboxyl groups, but not the protected primary amine groups, of the reaction product of step (a).

In some embodiments, which can be used in conjunction with the above aspects and embodiments, the multifunctional moiety comprises a residue of a polycarboxylamine compound comprising an amine group and n carboxyl groups, the amine group being used to form the amide group that links the multifunctional moiety to the amine-protected peptide residue, and the n carboxyl groups being used in the ester coupling reaction between the multifunctional precursor compound and the hydroxy-functional iodinated compound that comprises the iodinated moiety.

In some embodiments, which can be used in conjunction with the above aspects and embodiments, the multifunctional moiety comprises a residue of a polycarboxylamine compound comprising an amine group and n carboxyl groups, the amine group being used to form the amide group that links the multifunctional moiety to the amine-protected peptide residue, and the n carboxyl groups being used in the amide coupling reaction between the multifunctional precursor compound and the amine-functional iodinated compound that comprises the iodinated moiety.

In some embodiments, which can be used in conjunction with the above aspects and embodiments, the multifunctional moiety is linked to the amine-protected peptide residue through the ester group and the multifunctional moiety comprises the n carboxyl groups.

In some of these embodiments, the n carboxyl groups of the multifunctional precursor compound are reacted with n hydroxyl-functional iodinated compounds in an ester coupling reaction to form the iodinated, amine-protected peptide compound.

In some of these embodiments, the n carboxyl groups of the multifunctional precursor compound are reacted with n amine-functional iodinated compounds in an amide coupling reaction to form the iodinated, amine-protected peptide compound.

In some embodiments, which can be used in conjunction with the above aspects and embodiments, the multifunctional precursor compound is formed by a process that comprises (a) reacting in an ester coupling reaction (i) a polyol compound having n+1 hydroxyl groups with (ii) a carboxyl group at a C-terminus of the amine-protected peptide that comprises the two or more amino acid residues with the primary-amine-containing side group, the primary amine groups of the amine-protected peptide being protected from participation in the amide coupling reaction, the reaction product having n hydroxyl groups; and (b) reacting the n hydroxyl groups of the reaction product of step (a) with a cyclic anhydride in a ring opening reaction, thereby forming the n carboxyl groups at the sites of the n hydroxyl groups.

In some embodiments, which can be used in conjunction with the above aspects and embodiments, the multifunctional moiety comprises a residue of a polyol compound having n+1 hydroxyl groups, one of the n+1 hydroxyl amine groups being used to form the ester group that links the multifunctional moiety to the amine-protected peptide residue, n of the n+1 hydroxyl groups being used in a ring opening reaction with a cyclic anhydride to form the multifunctional moiety that comprises n carboxyl groups, and the n carboxyl groups being used in the ester coupling reaction between the multifunctional precursor compound and the hydroxy-functional iodinated compound that comprises the iodinated moiety.

In some embodiments, which can be used in conjunction with the above aspects and embodiments, the multifunctional moiety comprises a residue of a polyol compound having n+1 hydroxyl groups, one of the n+1 hydroxyl amine groups being used to form the ester group that links the multifunctional moiety to the amine-protected peptide residue, n of the n+1 hydroxyl groups being used in a ring opening reaction with a cyclic anhydride to form the multifunctional moiety that comprises n carboxyl groups, and the n carboxyl groups being used in the amide coupling reaction between the multifunctional precursor compound and the amine-functional iodinated compound that comprises the iodinated moiety.

In some embodiments, which can be used in conjunction with the above aspects and embodiments, the multifunctional precursor compound is formed by a process that comprises (a) reacting in an ester coupling reaction (i) a carboxyl-protected hydroxycarboxylic acid compound comprising a hydroxyl group and n protected carboxyl groups that are protected from reacting in the ester coupling reaction with (ii) a carboxyl group at a C-terminus of an amine-protected peptide that comprises the two or more amino acid residues with the primary-amine-containing side groups, the primary amine groups of the amine-protected peptide being protected from reacting in the amide coupling reaction; and (b) deprotecting the n protected carboxyl groups, but not the protected primary amine groups, of the reaction product of step (a).

In some embodiments, which can be used in conjunction with the above aspects and embodiments, the multifunctional moiety comprises a residue of a hydroxycarboxylic acid compound that comprises a hydroxyl group and n carboxyl groups, the hydroxyl group being used to form the ester group that links the multifunctional moiety to the amine-protected peptide residue, and the n carboxyl groups being used in the ester coupling reaction between the multifunctional precursor compound and the hydroxy-functional iodinated compound that comprises the iodinated moiety.

In some embodiments, which can be used in conjunction with the above aspects and embodiments, the multifunctional moiety comprises a residue of a hydroxycarboxylic acid compound comprising a hydroxyl group and n carboxyl groups, the hydroxyl group being used to form the ester group that links the multifunctional moiety to the amine-protected peptide residue, and the n carboxyl groups being used in the amide coupling reaction between the multifunctional precursor compound and the amine-functional iodinated compound that comprises the iodinated moiety.

In some embodiments, which can be used in conjunction with the above aspects and embodiments, the n iodinated moieties comprise one or more iodinated aromatic groups.

In some embodiments, which can be used in conjunction with the above aspects and embodiments, the n iodinated moieties comprise one or more iodinated aromatic groups that comprise a monocyclic or multicyclic aromatic structure that is substituted with one or more iodine groups. In some of these embodiments, the monocyclic or multicyclic aromatic structure is further substituted with one or more hydroxyl groups and/or one or more substituents that comprise a C₁-C₄-hydroxyalkyl group. In some of these embodiments, the monocyclic or multicyclic aromatic structure is further substituted with one or more substituents that comprise a C₁-C₄-hydroxyalkyl group and wherein the C₁-C₄-hydroxyalkyl group comprises adjacent hydroxyl groups, in which case the adjacent hydroxyl groups may comprise acetal protection when the multifunctional precursor compound is reacted with the iodinated compound.

In some embodiments, which can be used in conjunction with the above aspects and embodiments, the primary amine groups of the peptide comprise a protective group selected from a tert-butoxycarbonyl group, a carboxybenzyl group, a trifluoroacetyl group, a 6-nitroveratryloxycarbonyl group and a 9-fluorenylmethoxycarbonyl group.

In other aspects of the present disclosure, crosslinked networks are provided that comprises a crosslinked reaction product of (a) a radiopaque peptide compound formed in accordance with any of the above aspects and embodiments and (b) a reactive polymer that comprises moieties that react with the primary amine groups of the radiopaque peptide compound to form covalent linkages.

In further aspects of the present disclosure, a system is provided that comprises (a) a first composition that comprises a radiopaque peptide compound formed in accordance with any of the above aspects and embodiments and (b) a second composition that comprises a reactive polymer that comprises moieties that react with the primary amine groups of the radiopaque peptide compound, wherein the system is configured to deliver the reactive polymer and the radiopaque peptide compound under conditions such that covalent crosslinks are formed between the reactive polymer and the radiopaque peptide compound.

The above and other aspects, embodiments, features and benefits of the present disclosure will be readily apparent from the following detailed description.

### BRIEF DESCRIPTION OF THE DRAWINGS

FIGS. 1A-1D schematically illustrate processes for forming multifunctional precursor compounds, in accordance with four embodiments of the present disclosure.

FIG. 2 schematically illustrate processes for forming carboxyl-functional multifunctional precursor compounds, in accordance with three embodiments of the present disclosure.

FIG. 2A schematically illustrates a process for forming a carboxyl-functional multifunctional precursor compound, in accordance with another embodiment of the present disclosure.

FIG. 3 schematically illustrates a process for forming a carboxyl-functional multifunctional precursor compound, in accordance with another embodiment of the present disclosure.

FIG. 4 schematically illustrates a process for forming an iodinated peptide compound, in accordance with an embodiment of the present disclosure.

FIG. 5 schematically illustrates a process of forming an amine-functional polyiodinated compound, which is then reacted with protected trilysine, followed by deprotection, to form an iodinated peptide compound, in accordance with an embodiment of the present disclosure.

FIG. 6A schematically illustrates acetal protection of adjacent hydroxyl groups of iodixanol, in accordance with an embodiment of the present disclosure.

FIG. 6B schematically illustrates a process for forming an iodinated peptide compound, in accordance with an embodiment of the present disclosure.

FIGS. 7A-7E schematically illustrates processes for forming iodinated peptide compounds, in accordance with further embodiments of the present disclosure.

FIG. 8 illustrates a delivery device, in accordance with an embodiment of the present disclosure.

FIG. 9 illustrates a delivery device, in accordance with another embodiment of the present disclosure.

### DETAILED DESCRIPTION

In various aspects, the present disclosure provides radiopaque peptides in which a radiopaque moiety comprising one or more types of radiopaque atoms is covalently linked at the C-terminus of the peptide.

Peptides for use in the present disclosure include peptides containing from 2 to 20 amino acid residues having primary-amine-containing side groups (i.e., amino acid residues having —NH₂ groups, including guanidino groups), for example, lysine, arginine, and/or ornithine amino acid residues (e.g., dilysine, trilysine, tetralysine, pentalysine, hexalysine, heptalysine, octalysine, nonalysine, decalysine, diornithine, triornithine, tetraornithine, pentaornithine, hexaornithine, heptaornithine, octaornithine, nonaornithine, decaornithine, diarginine, triarginine, tetraarginine, pentaarginine, hexaarginine, heptaarginine, octaarginine, nonaarginine, decaarginine, etc.). Peptides for use in the present disclosure may also include amino acids in addition to lysine, arginine, and/or ornithine, particularly those having side groups that do not react with carboxyl, amine or hydroxyl groups under amide or ester coupling conditions. Examples of such peptides include glycine, alanine, valine, leucine, isoleucine, and phenylalanine, among others. Overall length of the peptides of the present disclosure typically range from 2 to 20 or more amino acid resides, for example, ranging from 2 to 3 to 4 to 5 to 6 to 8 to 10 to 15 to 20 amino acid resides (i.e., ranging between any two of the preceding values).

Peptides for use in the processes of the present disclosure include amine-protected peptides in which the amine at the N-terminus of the peptide and the primary-amine-containing side groups of the amino acid residues of the peptide are protected.

Radiopaque moieties comprising the one or more types of radiopaque atom(s) include iodinated moieties, which can comprise one, two, three, four, five, six, seven, eight, nine, ten, eleven, twelve or more iodine atoms.

In some embodiments, the iodinated moieties comprise one or more iodinated aromatic groups. Examples of iodinated aromatic groups include iodine-substituted monocyclic aromatic groups and iodine-substituted multicyclic aromatic groups, such as iodinated phenyl groups, iodinated naphthyl groups, iodinated anthracenyl groups, iodinated phenanthrenyl groups, or iodinated tetracenyl groups. The iodinated aromatic groups may be substituted with one, two, three, four, five, six, or more iodine atoms. In some of these embodiments, the aromatic groups may be further substituted with one or more hydrophilic groups, for example, one, two, three, four, five, six or more hydrophilic groups. The hydrophilic groups may be hydroxyl-containing groups, which may be selected, for example, from hydroxyl groups and hydroxyalkyl groups (e.g., hydroxyalkyl groups containing one carbon, two carbons, three carbons, four carbons, etc.).

Specific examples of iodinated moieties include those that comprise one or more monocyclic or multicyclic aromatic structures, substituted with (a) one or more iodine groups (e.g., one two, three, four, five, six, seven, eight, nine, ten or more iodine atoms) and (b) optionally, one or more hydroxyl-containing groups independently selected from one or more hydroxyl groups and/or one or more C₁-C₄-hydroxyalkyl groups (e.g., C₁-C₄-monohydroxyalkyl groups, C₁-C₄-dihydroxyalkyl groups, C₁-C₄-trihydroxyalkyl groups, C₁-C₄-tetrahydroxyalkyl groups, etc.), among others, which C₁-C₄-hydroxyalkyl groups may be linked to the monocyclic or multicyclic aromatic structures directly or through any suitable linking moiety, which may be selected, for example, from alkyl groups, ether groups, ester groups, amide groups, amine groups, carbonate groups, and combinations thereof, among others.

In some aspects of the present disclosure, radiopaque peptide compounds are formed by a method that comprises (a) forming a multifunctional precursor compound that comprises (i) a residue of an amine-protected peptide that comprises two or more amino acid residues with primary-amine-containing side groups and (ii) a multifunctional moiety linked to the amine-protected peptide residue through an amide group or an ester group, the multifunctional moiety further comprising n primary amine groups, n hydroxyl groups, or n carboxyl groups, wherein n is an integer of 1 or more (e.g., 2, 3, 4, 5, 6, 7, 8, 9, 10 or more); (b) reacting the multifunctional precursor compound in an amide coupling reaction or an ester coupling reaction with an iodinated compound selected from an amine-functional iodinated compound that comprises an iodinated moiety, a carboxyl-functional iodinated compound that comprises an iodinated moiety, and a hydroxy-functional iodinated compound that comprises an iodinated moiety, to form an iodinated, amine-protected peptide compound comprising n iodinated moieties that are covalently attached to the amine-protected peptide residue; and (c) deprotecting the iodinated, amine-protected peptide compound to form an iodinated peptide compound.

Examples of protective groups for use in conjunction with amide coupling reactions and ester coupling reactions include tert-butoxycarbonyl (Boc) groups, carboxybenzyl (Cbz) or (Z) groups, trifluoroacetyl (TFA) groups, 6-nitroveratryloxycarbonyl (Nvoc) groups, and 9-fluorenylmethoxycarbonyl (Fmoc) groups, or allyloxycarbonyl (Alloc) groups; trityl (Trt) groups, t-butyl (t-Bu) groups, among others.

In some embodiments, the multifunctional precursor compound comprises an amine-protected peptide residue and a multifunctional moiety that comprises n primary amine groups linked to the amine-protected peptide residue through an amide group.

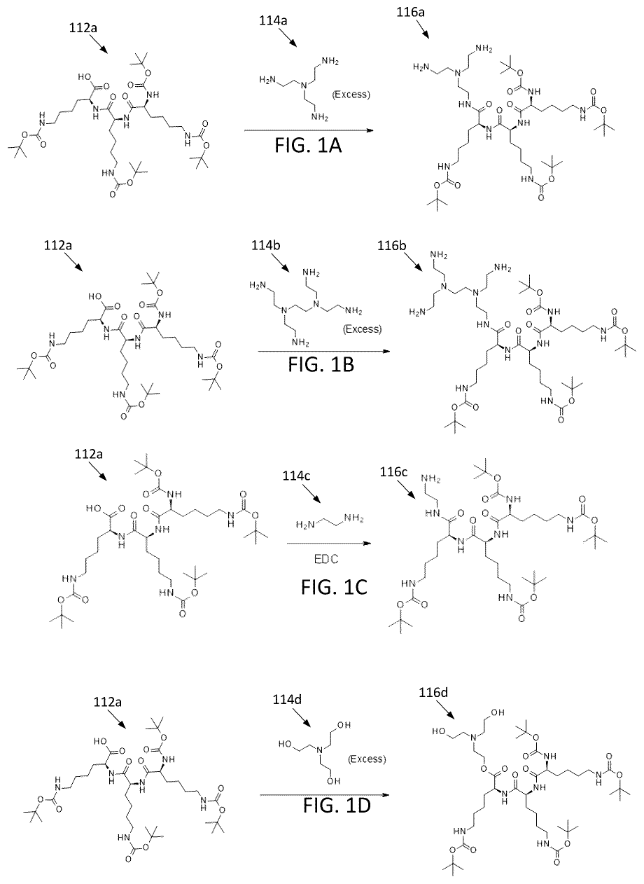

For example, with reference now to FIG. 1A, an amine-protected peptide, specifically, tert-butoxycarbonyl-protected trilysine (Boc-protected trilysine) (112a), is reacted in an amide coupling reaction with a polyamine compound having n+1 primary amine groups, specifically, tris(2-aminoethyl)amine,

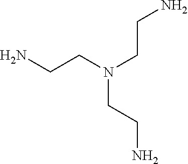

(CAS #4097-89-6) (114a) having three primary amine groups, to form a multifunctional precursor compound (116a) that comprises a Boc-protected trilysine residue and a multifunctional moiety having n primary amine groups, specifically, a tris(2-aminoethyl)amine residue having two primary amine groups, where the multifunctional moiety is linked to the amine-protected peptide residue through an amide group.

It is noted that the various amide coupling reactions and ester coupling reactions described herein may be performed in the presence of a suitable coupling agent, for example, a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another suitable coupling agent.

As another example, and with reference now to FIG. 1B, an amine-protected peptide, specifically, Boc-protected trilysine (112a), is reacted in an amide coupling reaction with a polyamine compound having n+1 primary amine groups, specifically, N,N′,N′-tetrakis(2-aminoethyl)-1,2-ethanediamine,

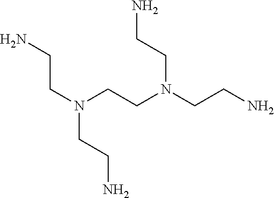 [SMILES: NCCN(CCN)CCN(CCN)CCN]

(CAS #4097-90-9) (114b) having four primary amine groups to form a multifunctional precursor compound (116b) that comprises a Boc-protected trilysine residue and a multifunctional moiety having n primary amine groups, specifically, a N,N′,N′-tetrakis(2-aminoethyl)-1,2-ethanediamine residue having three primary amine groups, where the multifunctional moiety is linked to the amine-protected peptide residue through an amide group.

As another example, and with reference now to FIG. 1C, an amine-protected peptide, specifically, Boc-protected trilysine (112a), is reacted in an amide coupling reaction with a polyamine compound having n+1 primary amine groups, specifically, ethylene diamine,

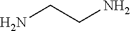

(CAS #107-15-3) (114c) having two primary amine groups to form a monofunctional precursor compound (116c) that comprises a Boc-protected trilysine residue and a monofunctional moiety having n primary amine groups, specifically, an ethylene diamine residue having one primary amine group, where the monofunctional moiety is linked to the amine-protected peptide residue through an amide group.

Various polyamine compounds can be used in conjunction with the present disclosure beyond the tris(2-aminoethyl)amine and N,N′,N′-tetrakis(2-aminoethyl)-1,2-ethanediamine of FIGS. 1A and 1B, including 3-(2-aminoethyl) pentane-1,5-diamine,

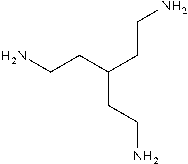

### (CAS #460078-00-6),

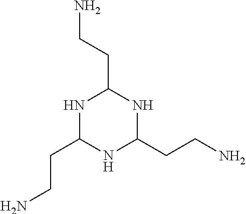 [SMILES: NCCC1NC(CCN)NC(CCN)N1]

(CAS #146117-64-8), 1,3,5-tris-(2-aminoethyl)-[1,3,5]triazinane-2,4,6-trione,

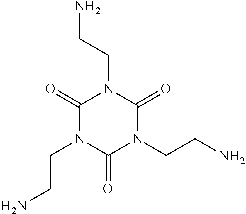 [SMILES: NCCn1c(=O)n(CCN)c(=O)n(CCN)c1=O]

(CAS #43190-26-7), N,N,N′-Tris(2-aminoethyl)ethylenediamine,

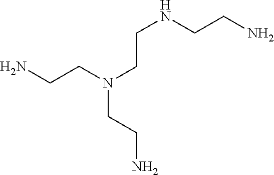 [SMILES: NCCNCCN(CCN)CCN]

(CAS #31295-46-2), and adamantane-1,3,5,7-tetraamine,

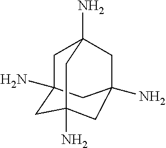 [SMILES: NC12CC3(N)CC(N)(C1)CC(N)(C2)C3]

### CAS #(16004-77-6).

Additional polyamine compounds can be found in Table 1 to follow.

TABLE 1

| | | # of amine |
|---|---|---|
| | | functional groups |
| Name | CAS# | in product |
| Polyethylene glycol bis(2- | 24991-53-5 | 1 |
| aminoethyl) ether | | |
| 1,3-Propanediamine | 109-76-2 | 1 |
| Tris(3-aminopropyl)amine | 4963-47-7 | 2 |
| 2,2-Bis(aminomethyl)-1,3- | 4742-00-1 | 3 |
| propanediamine | | |
| 1,4,7,10-Tetraazacyclododecane | 294-90-6 | 3 |
| Hexacyclen | 296-35-5 | 5 |

In various embodiments, the polyamine compounds do not contain a carboxylic acid group, which would require a suitable protection strategy. For example, in the event the polyamine contains a carboxylic acid group, it should be converted, for example, to a methyl ester.

In some embodiments, the multifunctional precursor compound comprises an amine-protected peptide residue and a multifunctional moiety that comprises n hydroxyl groups linked to the amine-protected peptide residue through an ester group.

For example, with reference now to FIG. 1D, an amine-protected peptide, specifically, Boc-protected trilysine (112a), is reacted in an ester coupling reaction with an polyol compound having n+1 hydroxy groups, specifically, triethanolamine,

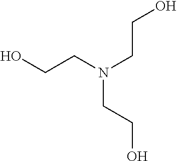

(CAS #102-71-6) (114d) having three hydroxy groups to form a multifunctional precursor compound (116d) that comprises a Boc-protected trilysine residue and a multifunctional moiety having n hydroxyl groups, specifically, a triethanolamine residue having two hydroxyl groups, where the multifunctional moiety is linked to the amine-protected peptide residue through an ester group.

Various polyol compounds can be used in conjunction with the present disclosure beyond the triethanolamine of FIG. 1D, including 3-(2-hydroxyethyl) pentane-1,5-diol,

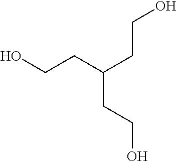

(CAS #53378-75-9), 1-[N,N-bis(2-hydroxyethyl)amino]-2-propanol,

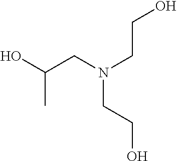

(CAS #6712-98-7), s-triazine-1,3,5-triethanol,

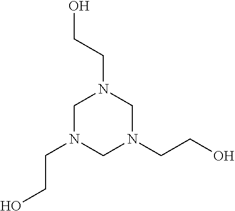 [SMILES: OCCN1CN(CCO)CN(CCO)C1]

(CAS #4719-04-4), tris(2-hydroxyethyl) isocyanurate,

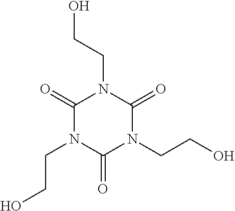 [SMILES: O=c1n(CCO)c(=O)n(CCO)c(=O)n1CCO]

(CAS #839-90-7), N,N,N′,N′-tetrakis(2-hydroxyethyl)ethylenediamine,

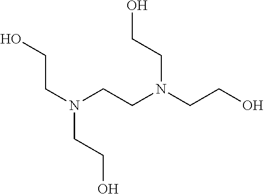 [SMILES: OCCN(CCO)CCN(CCO)CCO]

(CAS #140-07-8), miglitol,

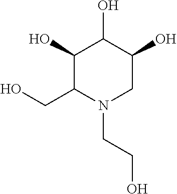 [SMILES: OCCN1C[C@H](O)C(O)[C@H](O)C1CO]

(CAS #7243203-2), and bis(2-hydroxyethyl)amino-tris(hydroxymethyl)methane,

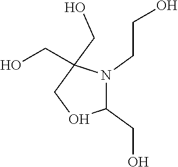 [SMILES: OCCN1C(CO)OCC1(CO)CO]

(CAS #6976-37-0), among others. Additional polyol compounds may be selected from suitable members of the core polyols described below. In various embodiments, the polyol compounds do not contain a carboxylic acid group, which would require a suitable protection strategy. For example, in the event the polyol contains a carboxylic acid group, it should be converted, for example, to a methyl ester.

In some embodiments, the multifunctional precursor compound comprises an amine-protected peptide residue and a multifunctional moiety that comprises n carboxyl groups linked to the amine-protected peptide residue through an amide group.

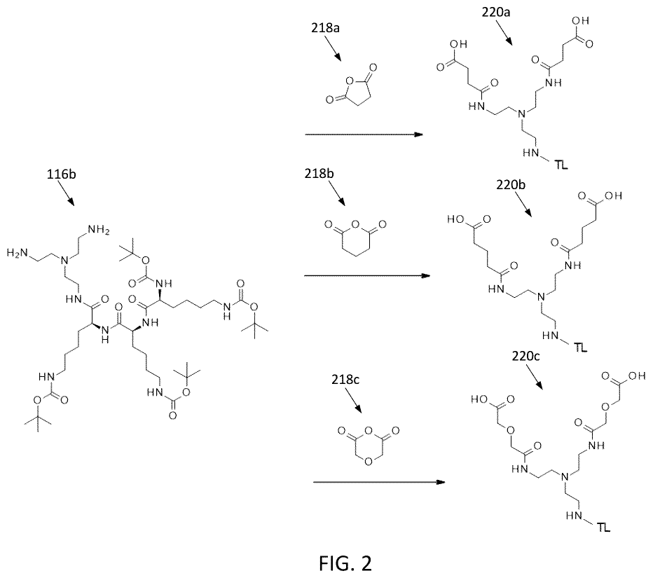

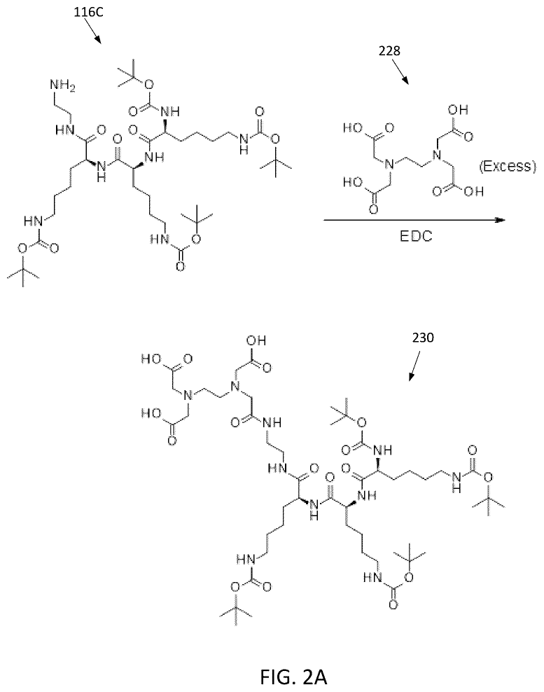

For example, with reference now to FIG. 2, a multifunctional compound (116a) that comprises a Boc-protected trilysine residue and a multifunctional moiety having n primary amine or hydroxyl groups, specifically, a tris(2-aminoethyl)amine residue having two primary amine groups as formed in FIG. 1A, is reacted with a cyclic anhydride, specifically, succinic anhydride,

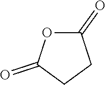

(CAS #108-30-5) (218a), in a ring opening reaction to form a multifunctional precursor compound (220a) that comprises a Boc-protected trilysine residue (represented by TL) and a multifunctional moiety having n carboxyl groups, specifically, a tris(2-aminoethyl)amine residue modified by two succinic anhydride residues providing two carboxyl groups, where the multifunctional moiety is linked to the amine-protected peptide residue through an amide group.

As another example, with continued reference to FIG. 2, a multifunctional compound (116a) that comprises a Boc-protected trilysine residue and a multifunctional moiety having two amine or hydroxyl groups, specifically, a tris(2-aminoethyl)amine residue, is reacted with a cyclic anhydride, specifically, glutaric anhydride,

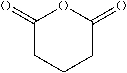

(CAS #108-55-4) (218b), in a ring opening reaction to form a multifunctional precursor compound (220b) that comprises a Boc-protected trilysine residue (represented by TL) and a multifunctional moiety having two carboxyl groups, specifically, a tris(2-aminoethyl)amine residue modified by two glutaric anhydride residues, where the multifunctional moiety is linked to the amine-protected peptide residue through an amide group.

As a further example, with further reference to FIG. 2, a multifunctional compound (116a) that comprises a Boc-protected trilysine residue and a multifunctional moiety having two amine or hydroxyl groups, specifically, a tris(2-aminoethyl)amine residue, is reacted with a cyclic anhydride, specifically, diglycolic anhydride,

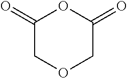

(CAS #4480-83-5) (218c), in a ring opening reaction to form a multifunctional precursor compound (220c) that comprises a Boc-protected trilysine residue (represented by TL) and a multifunctional moiety having two carboxyl groups, specifically, a tris(2-aminoethyl)amine residue modified by two diglycolic anhydride residues, where the multifunctional moiety is linked to the amine-protected peptide residue through an amide group.

Additional cyclic anhydrides are listed in Table 2. The cyclic anhydrides can be iodinated to provide more radiopacity.

TABLE 2

| Species | CAS # |
|---|---|
| Succinic Anhydride | 108-30-5 |
| Glutaric Anhydride | 108-55-4 |
| Adipic Anhydride | 2035-75-8 |
| 2,8-Oxocanedione | 10521-07-0 |
| 3-Oxabicyclo[3.1.0]hexane-2,4-dione | 5617-74-3 |
| 3-Oxabicyclo[3.2.0]heptane-2,4-dione | 4462-96-8 |
| Dihydro-4-methyl-2H-pyran-2,6(3H)-dione | 4166-53-4 |
| Itaconic anhydride | 2170-03-8 |
| 2,2-Dimethylsuccinic anhydride | 17347-61-4 |
| 6,6-Dimethyl-3-oxabicyclo[3.1.0]hexane-2,4-dione | 5466-84-2 |
| Dihydro-4,4-dimethyl-2H-pyran-2,6(3H)-dione | 4160-82-1 |
| Diglycolic anhydride | 4480-83-5 |
| 2H-Pyran-2,4,6(3H,5H)-trione | 10521-08-1 |
| Dihydro-4-(2-methylpropyl)-2H-pyran-2,6(3H)-dione | 185815-59-2 |
| 3-Iodophthalic anhydride* | 28418-88-4 |
| Tetraiodophthalic anhydride* | 632-80-4 |
| 5-Iodo-1,3-isobenzofurandione* | 28418-89-5 |
| 5,6-Diiodo-1,3-isobenzofurandione* | 25834-17-7 |
| Dihydro-3-(2-iodophenyl)-2,5-furandione* | 887131-98-8 |

*Iodinated Compounds

As another example and with reference to FIG. 2A, a monofunctional compound (116c) that comprises a Boc-protected trilysine residue and a monofunctional moiety having n primary amine groups, specifically, an ethylene diamine residue having one primary amine group, which is linked to the amine-protected peptide residue through an amide group, as formed in FIG. 1C, is reacted with a polycarboxylic acid compound, specifically, ethylenediamine tetraacetic acid (228) in an amide coupling reaction in the presence of a carbodiimide coupling agent such as EDC to form a multifunctional precursor compound (230) that comprises a Boc-protected trilysine residue and a multifunctional moiety having n carboxyl groups, specifically, an ethylene diamine residue modified by a ethylenediamine tetraacetic acid residue having three carboxyl groups, where the multifunctional moiety is linked to the amine-protected peptide residue through an amide group.

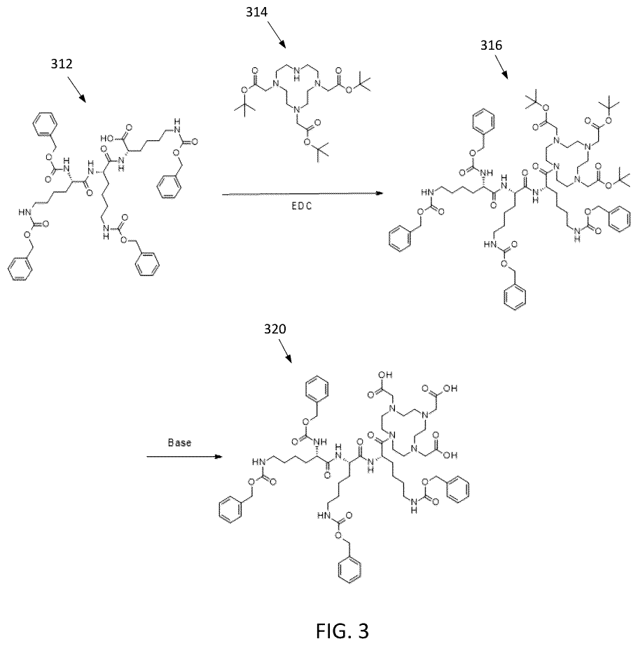

As another example and with reference to FIG. 3, an amine-protected peptide, specifically, carboxybenzyl-protected trilysine (Cbz-protected trilysine) (312), is reacted in an amide coupling reaction with a protected polycarboxylamine compound comprising at least one amine group and n protected carboxyl groups, specifically, 1,4,7,10-Tetraazacyclododecane-1,4,7-triacetic acid, 1,4,7-tris(1,1-dimethylethyl) ester,

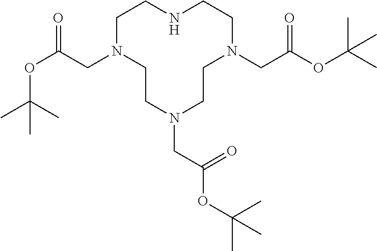 [SMILES: CC(C)(C)OC(=O)CN1CCNCCN(CC(=O)OC(C)(C)C)CCN(CC(=O)OC(C)(C)C)CC1]

(CAS #122555-91-3) (314), having one secondary amine group and three Boc-protected carboxyl groups, to form a protected intermediate compound (316). The Boc entities of the protected intermediate compound (316) are then deprotected with base to form a multifunctional precursor compound (320) that comprises a Cbz-protected trilysine residue and a multifunctional moiety having n carboxyl groups, specifically three carboxyl groups, where the multifunctional moiety is linked to the amine-protected peptide residue through an amide group.

Polyamines that have a carboxylic acid protected, which can be subsequently removed, include: β-Alanine, N-[3-(1,1-dimethylethoxy)-3-oxopropyl]-N-(2-hydroxyethyl)-, 1,1-dimethylethyl ester (CAS 831216-48-9), L-Glutamic acid di-tert-butyl ester (CAS 1687406-9), Heptanedioic acid, 4-amino-4-[3-(1,1-dimethylethoxy)-3-oxopropyl]-, 1,7-bis(1,1-dimethylethyl) ester (CAS 136586-99-7).

In further embodiments, the multifunctional precursor compound comprises an amine-protected peptide residue and a multifunctional moiety linked to the amine-protected peptide residue through an ester group, the multifunctional moiety further comprising n carboxyl groups.

As an example, a multifunctional compound such as compound (116d) of FIG. 1D, which comprises a Boc-protected trilysine residue and a multifunctional moiety having n hydroxyl groups, specifically, a triethanolamine residue having three hydroxyl groups, can be reacted with a cyclic anhydride, such as succinic anhydride, glutaric anhydride or diglycolic anhydride in a ring opening reaction to form a multifunctional precursor compound that comprises a Boc-protected trilysine residue and a multifunctional moiety having n carboxyl groups, specifically, a triethanolamine residue modified by two glutaric anhydride residues, two succinic anhydride residues, or two diglycolic anhydride residues, which provide two carboxyl groups, where the multifunctional moiety is linked to the amine-protected peptide residue through an ester group.

As another example, an amine-protected peptide, for example, carboxybenzyl-protected trilysine (e.g., Cbz-protected trilysine) (312) shown in FIG. 3 may be reacted in an ester coupling reaction with a carboxyl-protected hydroxycarboxylic acid compound comprising a hydroxyl group and n protected carboxyl groups, for example, Boc-protected citric acid having one hydroxyl group and three Boc-protected carboxyl groups, to form a protected intermediate compound. Then, analogous to FIG. 3, the Boc entities of the protected intermediate compound are deprotected with base to form a multifunctional precursor compound that comprises a Cbz-protected trilysine residue and a multifunctional moiety having n carboxyl groups, specifically three carboxyl groups, where the multifunctional moiety is linked to the amine-protected peptide residue through an ester group.

Polyols that have a carboxylic acid protected, which can be subsequently removed, include: 4,10,14-Trioxa-7-azahexadecanoic acid, 7-(2-hydroxyethyl)-15,15-dimethyl-13-oxo-, 1,1-dimethylethyl ester (CAS 1415800-34-8), Glycine, N-[2-(1,1-dimethylethoxy)-2-oxoethyl]-N-(2-hydroxyethyl)-, 1,1-dimethylethyl ester (CAS 146432-41-9).

As previously indicated, once a multifunctional precursor compound such as any of those described above, among others, is provided, the multifunctional precursor compound may be reacted with an amine-functional iodinated compound, a carboxyl-functional iodinated compound, or hydroxy-functional iodinated compound, in an ester coupling reaction or an amide coupling reaction, to form a protected compound in which n iodinated moieties are attached to the amine-protected peptide residue, where, as previously noted, n is an integer of 2 or more (e.g., 2, 3, 4, 5, 6, 7, 8, 9, 10 or more).

For example, (a) a multifunctional precursor compound comprising an amine-protected peptide residue and a multifunctional moiety comprising n carboxyl groups can be reacted in an amide coupling reaction with n amine-functional iodinated compounds that comprise an iodinated moiety to form a protected compound in which n iodinated moieties are linked to the amine-protected peptide residue through amide linkages, (b) a multifunctional precursor compound comprising an amine-protected peptide residue and a multifunctional moiety comprising n carboxyl groups can be reacted in an ester coupling reaction with n hydroxy-functional iodinated compounds that comprise an iodinated moiety to form a protected compound in which n iodinated moieties are linked to the amine-protected peptide residue through ester linkages, (c) a multifunctional precursor compound comprising an amine-protected peptide residue and a multifunctional moiety comprising n primary amine groups can be reacted in an amide coupling reaction with n carboxyl-functional iodinated compounds that comprise an iodinated moiety to form a protected compound in which n iodinated moieties are linked to the amine-protected peptide residue through amide linkages, or (d) a multifunctional precursor compound comprising an amine-protected peptide residue and a multifunctional moiety comprising n hydroxyl groups can be reacted in an ester coupling reaction with n carboxyl-functional iodinated compounds that comprise an iodinated moiety to form a protected compound in which n iodinated moieties are linked to the amine-protected peptide residue through ester linkages.

After forming the protected compound in which n iodinated moieties are linked to the amine-protected peptide residue, the protective groups may then be removed. For example, Boc-protected groups may be deprotected by exposure to acid (e.g., hydrochloric acid or trifluoroacetic acid), Cbz protected groups may be deprotected by using the reducing agent such as NaBH₄ and catalytic Pd—C in methanol, while taking caution not to remove hydrogenate the iodinated rings, and so forth.

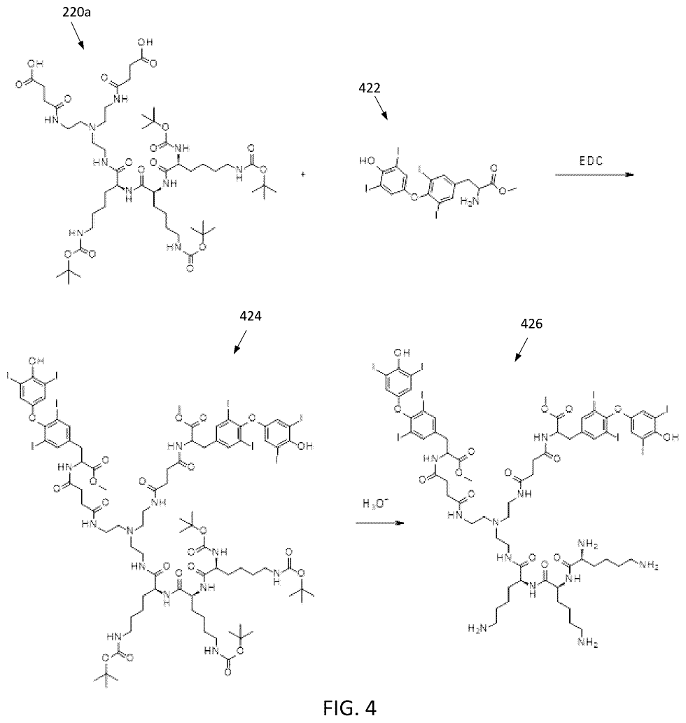

As a specific example, with reference to FIG. 4, a multifunctional precursor compound, specifically, multifunctional precursor compound (220a) like described in FIG. 2, which comprises a Boc-protected trilysine residue and a multifunctional moiety having n carboxyl groups, specifically, two carboxyl groups, is reacted with n molecules of an amine-functional iodinated compound that comprises an iodinated moiety, specifically, two molecules of tetraiodothyronine methyl ester, also known as thyroxine methyl ester,

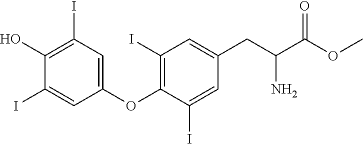 [SMILES: COC(=O)C(N)Cc1cc(I)c(Oc2cc(I)c(O)c(I)c2)c(I)c1]

(CAS #3218011-3) (422), in an amide coupling reaction where the carboxyl groups of the multifunctional precursor compound (220a) are reacted with the amine group of the thyroxine methyl ester (422) to form an amide linkage. The resulting compound is a protected compound in which n iodinated moieties are attached to an amine-protected peptide residue, specifically, a protected iodinated peptide compound (424) comprising two thyroxine methyl ester residues and a Boc-protected trilysine residue. In this particular embodiment, the two thyroxine methyl ester residues are connected to the Boc-protected trilysine residue through a linkage that comprises a residue of a polyamine compound, specifically, a tris(2-aminoethyl)amine residue. The Boc-protection is then removed to provide an iodinated peptide compound (426) comprising two thyroxine methyl ester residues and a trilysine residue. In this particular embodiment, the two thyroxine methyl ester residues are connected to the trilysine residue through a linkage that comprises a residue of a polyamine compound, specifically, a tris(2-aminoethyl)amine residue.

Other examples of amine-functional iodinated compounds, several of which are iodinated amino acid esters, include monoiodo-phenylalanine methyl ester,

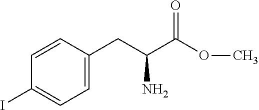

(CAS #158686-46-5), monoiodotyrosine ethyl ester,

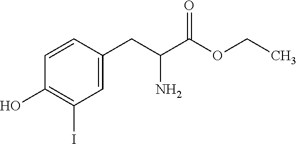 [SMILES: CCOC(=O)C(N)Cc1ccc(O)c(I)c1]

(CAS #10051-55-5), diiodotyrosine methyl ester,

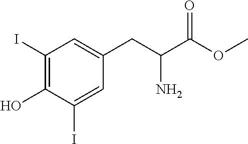 [SMILES: COC(=O)C(N)Cc1cc(I)c(O)c(I)c1]

(CAS #21959-36-4), dijodotyrosine ethyl ester,

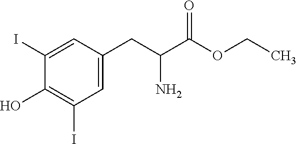 [SMILES: CCOC(=O)C(N)Cc1cc(I)c(O)c(I)c1]

(CAS #74051-47-1), diiodothyronine methyl ester,

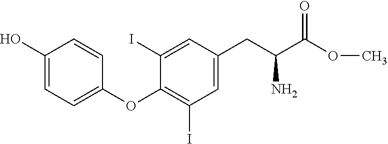 [SMILES: COC(=O)[C@@H](N)Cc1cc(I)c(Oc2ccc(O)cc2)c(I)c1]

(CAS #203585-45-9), triiodothyronine methyl ester, also known as T3 methyl ester,

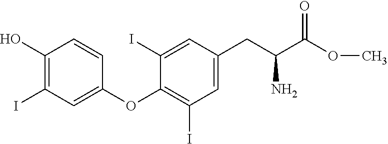 [SMILES: COC(=O)[C@@H](N)Cc1cc(I)c(Oc2ccc(O)c(I)c2)c(I)c1]

(CAS #3005-96-7), 5-amino-N,N′-bis(2,3-dihydroxypropyl)-2,4,6-triiodoisophthalamide (also known as iohexol related compound J),

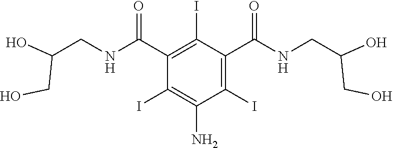 [SMILES: Nc1c(I)c(C(=O)NCC(O)CO)c(I)c(C(=O)NCC(O)CO)c1I]

(CAS #CAS 76801-93-9), acetal-protected 5-amino-N,N′-bis(2,3-dihydroxypropyl)-2,4,6-triiodoisophthalamide (acetal-protected iohexol related compound J),

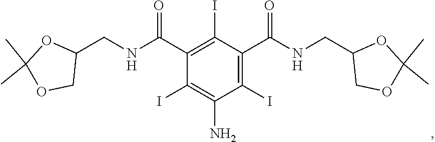

5-amino-N,N′-bis(2,3-dihydroxypropyl)-2,4,6-triiodoisophthalamide (also known as Iohexol EP Impurity F),

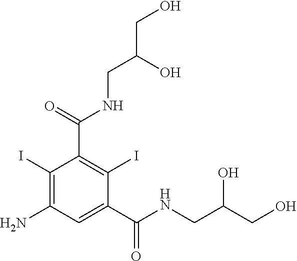 [SMILES: Nc1cc(C(=O)NCC(O)CO)c(I)c(C(=O)NCC(O)CO)c1I]

(CAS #1215856-35-1), acetal-protected Iohexol EP Impurity F, dimethyl 5-amino-2,4,6-triiodo-1,3-benzenedicarboxylate,

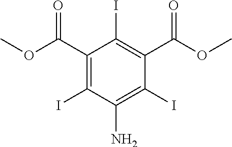

(CAS #15492111-6), 2,4,6-triiodoanaline,

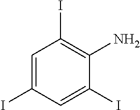

(CAS #24154-37-8), and triiodobenzylamine,

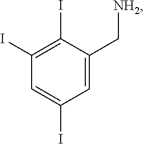 [SMILES: NCc1cc(I)cc(I)c1I]

among others.

In a further example, an amine-functional iodinated compound can be formed by reacting an amine-protected polycarboxylated amine compound having a protected amine group and m carboxyl groups, wherein m is an integer of 2 or more (e.g., 2, 3, 4, 5, 6, 7, 8, 9, 10 or more) with m amine-functional iodinated compounds that comprise an iodinated moiety in an amide coupling reaction. Deprotection of the amine group of the resulting product yields an amine-functional iodinated compound comprising an amine group and m iodinated moieties, more particularly, an amine-functional iodinated compound comprising a residue of a polycarboxylated amine compound and m amine-functional iodinated compound residues. Examples of polycarboxylated amine compounds include glutamic acid (CAS #56-86-0), 4-amino-4-(2-carboxyethyl)heptanedioic acid (CAS #176738-98-0), N-(5-amino-1-carboxypentyl)iminodiacetic acid (CAS #11323105-3), glutamyl-glutamic acid (CAS #3929-61-1), triglutamic acid (CAS #23684-48-2), or N2,N2-bis(carboxymethyl)lysine (CAS #129179-17-5).

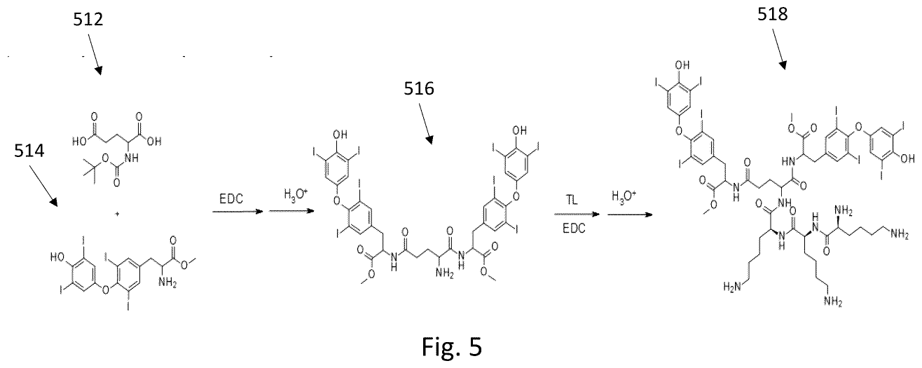

In a particular embodiment shown schematically in FIG. 5, an amine-protected polycarboxylated amine compound, specifically, a tert-butoxycarbonyl-protected glutamic acid (Boc-protected glutamic acid) (512) having two carboxylate groups and one protected amine group, is reacted in an amide coupling reaction with two molecules of an amine-functional iodinated compound that comprise an iodinated moiety, specifically, two molecules of thyroxine methyl ester (514), in the presence of a suitable coupling agent, for example, 1-ethyl-3-(3-dimethylpropyl)carbodiimide (EDC), to form an amine-protected compound. Deprotection of the amine group with acid yields an amine-functional iodinated compound (516) comprising a comprising two thyroxine methyl ester residues and a glutamic acid residue.

The amine group of the amine-functional iodinated compound comprising an amine group and m iodinated moieties can then be reacted, in an amide coupling reaction, with a carboxyl group of an amine-protected peptide that comprises two or more amino acid residues having primary-amine-containing side groups to form an iodinated, amine-protected peptide compound comprising m iodinated moieties that are covalently attached to a residue of the amine-protected peptide through a residue of the polycarboxylated amine compound. Deprotection of the amine group with acid yields an iodinated peptide compound comprising m iodinated moieties that are covalently attached to a residue of a peptide that comprises two or more amino acid residues having primary-amine-containing side groups through a residue of the polycarboxylated amine compound.

For example, turning again to the particular embodiment shown schematically in FIG. 5, amine-functional iodinated compound (516) is reacted in an amide coupling reaction with amine-protected trilysine (TL) in the presence of a suitable coupling agent, for example, EDC, to form a compound comprising two thyroxine methyl ester residues linked to an amine-protected trilysine residue through a glutamic acid residue. Deprotection of the amine group (e.g., with hydronium) yields an iodinated peptide compound (518) comprising two thyroxine methyl ester residues linked to a trilysine residue through a glutamic acid residue.

Alternatively, the amine group of the amine-functional iodinated compound comprising an amine group and m iodinated moieties can be reacted, in an amide coupling reaction, with an amine-protected multifunctional precursor compound as previously described which comprises (i) an amine-protected peptide residue that comprises two or more amino acid residues with primary-amine-containing side groups and (ii) a multifunctional moiety linked to the amine-protected peptide residue through an amide group or an ester group, the multifunctional moiety comprising n carboxyl groups, wherein n is an integer of 2 or more. Amine protection is then removed to yield an iodinated peptide compound that comprises n×m iodinated moieties that are connected to the peptide residue.

More particularly, the amine group of the amine-functional iodinated compound comprising a residue of a polycarboxylated amine compound and either (a) m amine-functional iodinated compound residues or (b) m hydroxy-functional iodinated compound residues can be reacted, in an amide coupling reaction, with carboxyl groups of an amine-protected multifunctional precursor compound which comprises (i) an amine-protected peptide residue that comprises two or more amino acid residues with primary-amine-containing side groups and (ii) a multifunctional moiety linked to the amine-protected peptide residue through an amide group or an ester group, the multifunctional moiety comprising n carboxyl groups, wherein n is an integer of 2 or more. Amine protection is then removed to yield an iodinated peptide compound that comprises n×m iodinated moieties that are covalently linked to the peptide residue, in particular, n×m iodinated moieties are connected to the peptide residue through n polycarboxylated amine compound residues and a residue of multifunctional the multifunctional moiety.

In a specific example, a multifunctional precursor compound, for example, a multifunctional precursor compound (220a), (220b) or (220c) like described in FIG. 2, which comprises a Boc-protected trilysine residue and a multifunctional moiety having n carboxyl groups, specifically, two carboxyl groups, can be reacted with n molecules of the amine-functional iodinated compound (516), which contains m thyroxine methyl ester residues, specifically two thyroxine methyl ester residues, and a glutamic acid residue, in an amide coupling reaction where the amine group of the amine-functional iodinated compound (516) is reacted with each of the carboxyl groups of the multifunctional precursor compound (220a), (220b) or (220c). After coupling, the Boc-protection is then removed to provide an iodinated peptide compound comprising n×m iodinated moieties, specifically, to provide an iodinated peptide compound comprising four thyroxine methyl ester residues and a trilysine residue. In this particular embodiment, the four thyroxine methyl ester residues are connected to the trilysine residue through a linkage that comprises a residue of a polyamine compound, specifically, a tris(2-aminoethyl)amine residue, two anhydride residues, and two glutamic acid residues.

Carboxyl-functional iodinated compounds for use in the present disclosure include triiodobenzoic acid

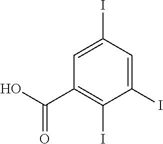 [SMILES: O=C(O)c1cc(I)cc(I)c1I]

(CAS #88-82-4), diatrizoic acid,

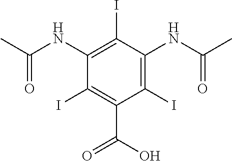 [SMILES: CC(=O)Nc1c(I)c(NC(C)=O)c(I)c(C(=O)O)c1I]

(CAS #117-96-4), N-acetyl-3,5-diiodo-L-tyrosine,

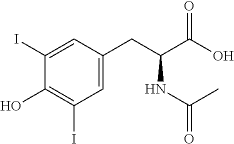 [SMILES: CC(=O)N[C@@H](Cc1cc(I)c(O)c(I)c1)C(=O)O]

(CAS #1027-28-7), N-acetyl-3-diiodo-L-tyrosine,

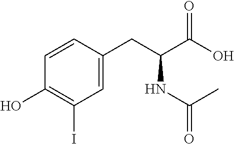 [SMILES: CC(=O)N[C@@H](Cc1ccc(O)c(I)c1)C(=O)O]

(CAS #1023-47-8) and N-acetyl-thyroxine,

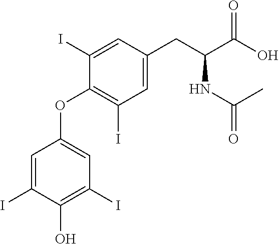 [SMILES: CC(=O)N[C@@H](Cc1cc(I)c(Oc2cc(I)c(O)c(I)c2)c(I)c1)C(=O)O]

(CAS #26041-51-0), among many others.

Additional carboxyl-functional iodinated compounds can be found in Table 3.

TABLE 3

| | | # I atoms |
|---|---|---|
| Name | CAS# | in product |
| 2-Iodobenzoic acid | 88-67-5 | 1 |
| 4-Iodobenzoic acid | 619-58-9 | 1 |
| 3-Iodobenzoic acid | 618-51-9 | 1 |
| 4-Iodopicolinic acid | 405939-79-9 | 1 |
| 3,5-Diiodobenzoic acid | 19094-48-5 | 2 |
| 3,4-Diiodobenzoic acid | 35674-20-5 | 2 |
| 3,5-Diiodo-4-(4- | 34043-77-1 | 2 |
| methoxyphenoxy)benzoic acid | | |
| Acetrizoic acid | 85-36-9 | 3 |
| Metrizoate | 1949-45-7 | 3 |
| 3,4,5-Triiodobenzoic acid | 2338-20-7 | 3 |
| Ioxitalamic acid | 28179-44-4 | 3 |
| Iothalamic acid | 2276-90-6 | 3 |
| 2,4,6-Triiodo-1,3,5- | 79211-41-9 | 3 |
| benzenetricarboxylic acid* | | |
| 2,4,6-Triiodobenzoic acid | 2012-31-9 | 3 |
| enzoic acid, 3-[[[2-hydroxy-1- | 87932-11-4 | 3 |
| (hydroxymethyl)ethyl]amino]carbonyl]- | | |
| 5-[(2-hydroxy-1-oxopropyl)amino]- | | |
| 2,4,6-triiodo-, (S)- (9CI) | | |
| Ioseric acid | 51876-99-4 | 3 |
| 4-[4-(Acetyloxy)-3-iodophenoxy]-3,5- | 2260-084 | 3 |
| diiodobenzoic acid | | |
| Ioglicic acid | 49755-67-1 | 3 |
| 2,4,6-Triiodo-3-[(1-oxo-3,6,9,12,15- | 16024-67-2 | 3 |
| pentaoxahexadec-1-yl)amino]benzoic acid | | |
| 3-[[(1,1- | 2358047-48-8 | 3 |
| Dimethylethoxy)carbonyl]amino]- | | |
| 2,4,6-triiodobenzoic acid | | |
| 2,3,4,6-Tetraiodobenzoic acid | 71463-71-3 | 4 |
| Tetraiodothyroformic Acid | 2055-97-2 | 4 |
| 2,3,5,6-Tetraiodo-1,4- | 7606-84-0 | 4 |
| benzenedicarboxylic acid* | | |
| D-Tyrosine, N-[(1,1- | 89624-64-6 | 4 |
| dimethylethoxy)carbonyl]-O-(4- | | |
| hydroxy-3,5-diiodophenyl)-3,5-diiodo- | | |
| (9CI, ACI) | | |
| 2,3,4,5,6-Pentaiodobenzoic acid | 64385-02-0 | 5 |
| Ioxaglic acid | 59017-64-0 | 6 |
| Ioglycamic acid* | 2618-25-9 | 6 |
| Iocarmic acid* | 10397-75-8 | 6 |

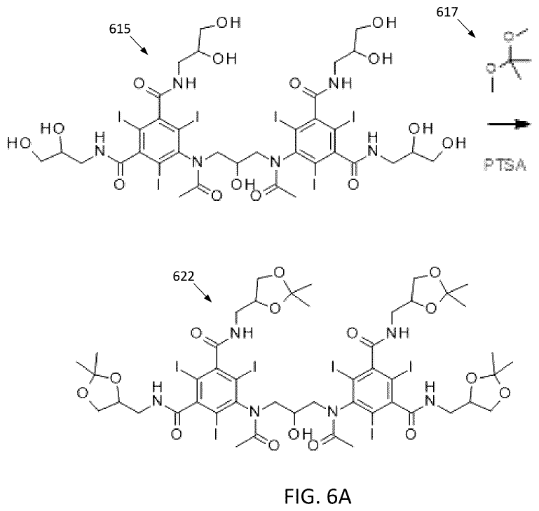

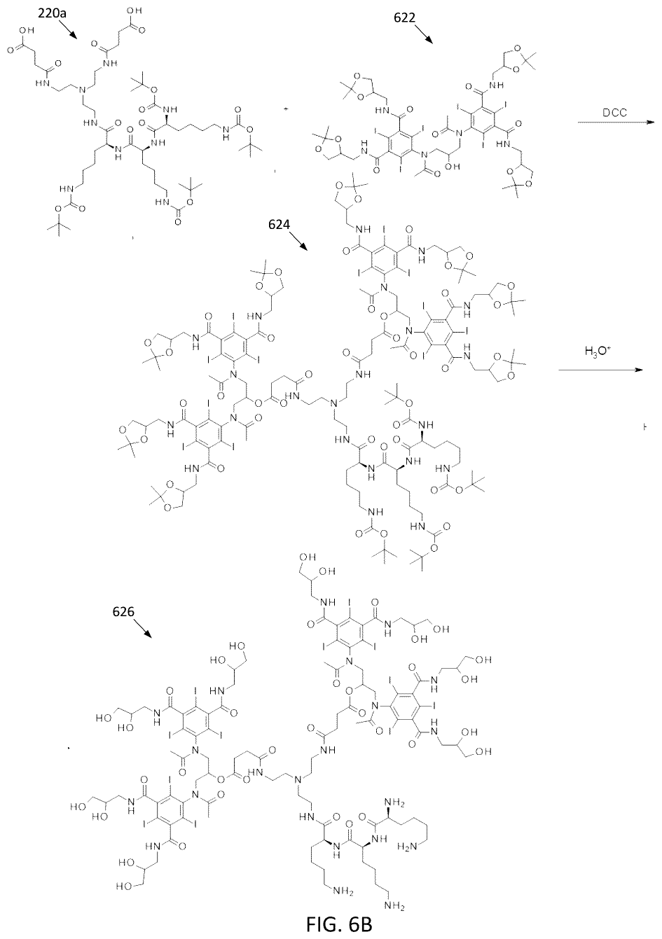

In some embodiments, functional groups in the amine-functional iodinated compound, carboxyl-functional iodinated compound, or hydroxy-functional iodinated compound that would undesirably participate in amide or ester coupling reactions may be protected. As one example, and with reference to FIG. 6A, adjacent hydroxyl groups of iodixanol (615) may be protected using 2,2-dimethoxypropane (617) in the presence of p-toluenesulfonic acid (PTSA) at room temperature, to obtain an acetal-protected hydroxy-functional iodinated compound, specifically, acetal-protected iodixanol (622).

The acetal-protected hydroxy-functional iodinated compound may then be coupled in an ester coupling reaction to a multifunctional precursor compound that comprises an amine-protected peptide residue and a multifunctional moiety comprising two or more carboxyl groups such as multifunctional precursor compounds (220a), (220b), (220c) of FIG. 2, or multifunctional precursor (320) of FIG. 3. For example, with reference to FIG. 6B, the acetal-protected iodixanol (622) produced in FIG. 6A may be coupled, in an ester coupling reaction, to the multifunctional precursor compound (220a) produced in FIG. 2 in the presence of a suitable coupling agent, for example, N,N′-dicyclohexylcarbodiimide (DCC) to form a protected intermediate product (624), which is then deprotected (e.g., with hydronium (H₃O⁺)) a provide an iodinated peptide compound (626) comprising two iodixanol residues and a trilysine residue. In this particular embodiment, the two iodixanol residues are connected to the trilysine residue through a linkage that comprises a tris(2-aminoethyl)amine residue.

In the case of iodixanol, it noted that an alternative scheme is possible in which the central hydroxyl group of the iodixanol is reacted, for example, with a diamine compound to provide an amine-functional iodinated compound having a primary amine group that is linked to an iodixanol residue by an amide-based linkage, which can participate in an amide coupling reaction with a multifunctional precursor compound that comprises an amine-protected peptide residue and a multifunctional moiety comprising two or more carboxyl groups.

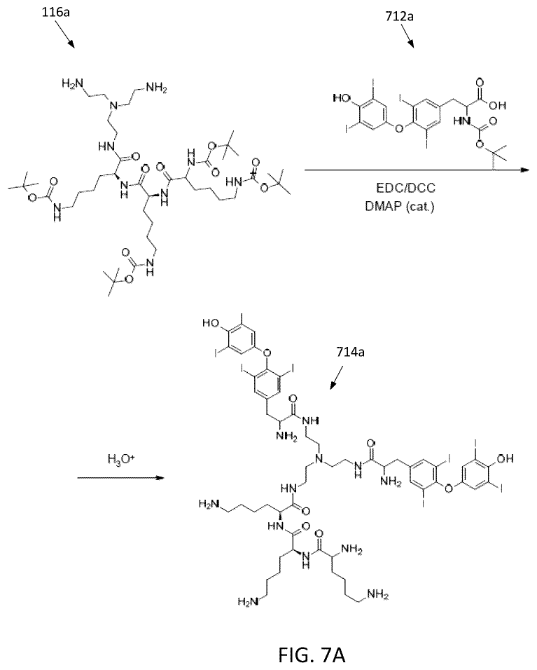

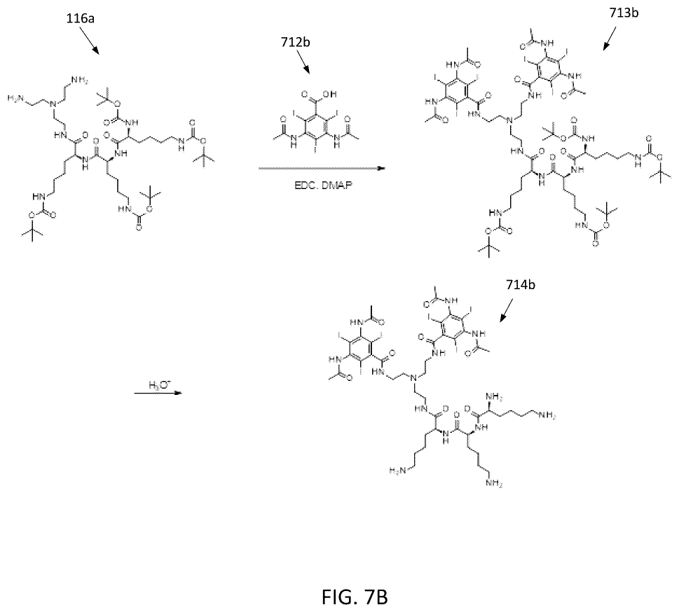

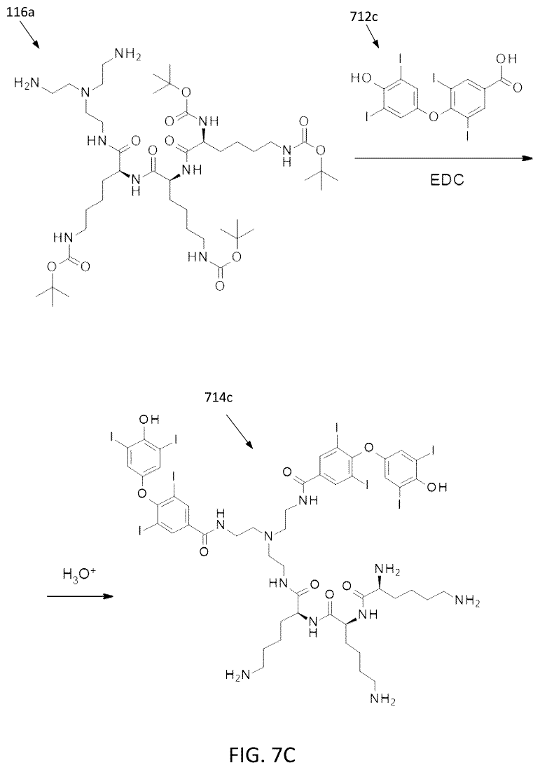

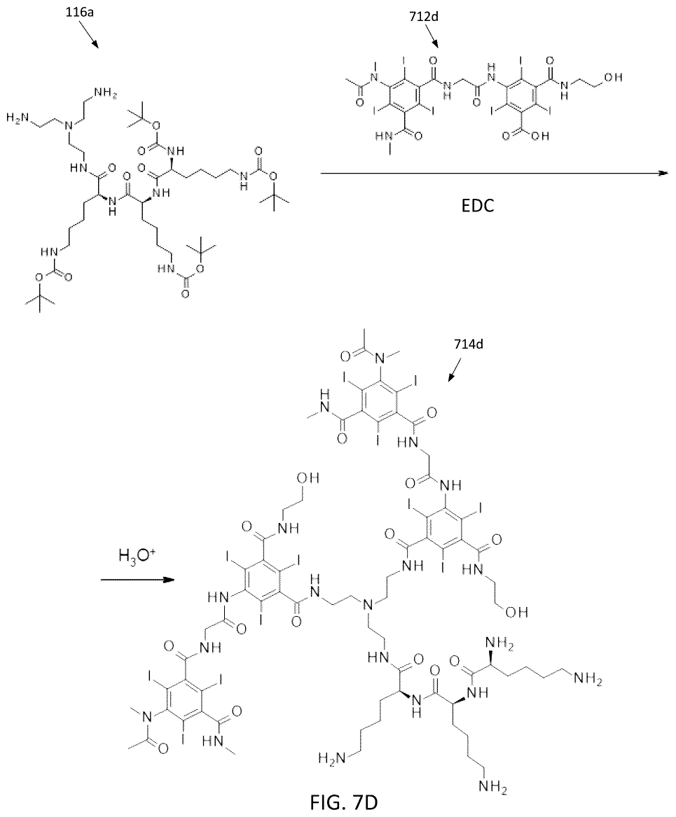

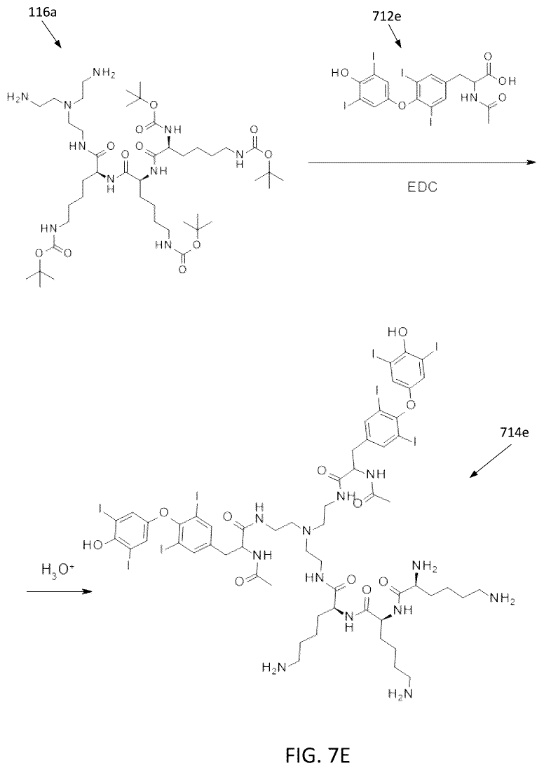

Another example, in which amine functional groups of an iodinated amino acid compound are protected, is described in conjunction with FIG. 7A. In FIG. 7A, a multifunctional precursor compound, specifically, a multifunctional precursor compound (116a) like described in FIG. 1A, which comprises a Boc-protected trilysine residue and a multifunctional moiety having n primary amine groups, specifically, a tris(2-aminoethyl)amine residue having two primary amine groups, is reacted with n molecules of an amine-protected iodinated amino acid compound, specifically, two molecules of Boc-protected thyroxine,

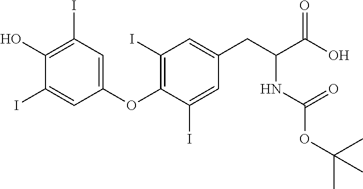 [SMILES: CC(C)(C)OC(=O)NC(Cc1cc(I)c(Oc2cc(I)c(O)c(I)c2)c(I)c1)C(=O)O]

(CAS #88404-22-2) (712a), in an amide coupling reaction where the amine groups of the multifunctional precursor compound (116a) are reacted with the carboxyl group of the Boc-protected thyroxine (712a) to form amide linkages. The amide coupling reaction shown is performed in the presence of a carbodiimide coupling agent, such as 1-ethyl-3-(3-dimethyl propyl) carbodiimide (EDC) or N,N′-dicyclohexylcarbodiimide (DCC), along with the use a catalytic amount of 4-dimethylaminopyridine (DMAP). The resulting compound is a protected compound in which n amine protected iodinated moieties are attached to an amine-protected peptide residue, specifically, a protected iodinated peptide compound comprising two Boc-protected thyroxine residues and a Boc-protected trilysine residue (not shown). In this particular embodiment, the two Boc-protected thyroxine residues are connected to the Boc-protected trilysine residue through a linkage that comprises a residue of a polyamine compound, specifically, a tris(2-aminoethyl)amine residue. The Boc-protection is then removed under acidic conditions to provide an iodinated peptide compound (714a) comprising two thyroxine residues and a trilysine residue. In this particular embodiment, the two thyroxine residues are connected to the trilysine residue through a linkage that comprises a residue of a polyamine compound, specifically, a tris(2-aminoethyl)amine residue.

It is noted that this process increases the number of primary amines from four to six, which will improve the water solubility and increase the number of primary amines that are available for future crosslinking. More generally, reaction between a primary-amine-protected iodinated compound with a multifunctional precursor compound comprising an amine-protected peptide residue will result an iodinated peptide compound having primary amine groups in addition to the primary amine groups associated with the peptide residue.

Particular embodiments where a carboxyl-functional iodinated compound is reacted with a multifunctional precursor compound comprising an amine-protected peptide residue and a multifunctional moiety comprising n primary amine groups, will now be described in conjunction with FIGS. 7B-7E.

Referring now to FIG. 7B, a multifunctional precursor compound, specifically, a multifunctional precursor compound (116a) like described in FIG. 1A, which comprises a Boc-protected trilysine residue and a multifunctional moiety having n primary amine groups, specifically, a tris(2-aminoethyl)amine residue having two primary amine groups, is reacted with n molecules of an amine-protected iodinated amino acid compound, specifically, two molecules of diatrizoic acid,

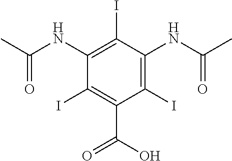 [SMILES: CC(=O)Nc1c(I)c(NC(C)=O)c(I)c(C(=O)O)c1I]

(712b), in an amide coupling reaction where the amine groups of the multifunctional precursor compound (116a) are reacted with the carboxyl group of the diatrizoic acid (712b) to form amide linkages. The amide coupling reaction shown is performed in the presence of a carbodiimide coupling agent, specifically, EDC. The resulting compound is a protected compound in which n iodinated moieties are attached to an amine-protected peptide residue, specifically, a protected iodinated peptide compound comprising two diatrizoic acid residues and a Boc-protected trilysine residue (not shown). The Boc-protection is then removed under acidic conditions to provide an iodinated peptide compound (714b) comprising diatrizoic acid residues and a trilysine residue. In this particular embodiment, the two diatrizoic acid residues are connected to the trilysine residue through a linkage that comprises a residue of a polyamine compound, specifically, a tris(2-aminoethyl)amine residue.

Referring now to FIG. 7C, a multifunctional precursor compound, specifically, a multifunctional precursor compound (116a) like described in FIG. 1A, which comprises a Boc-protected trilysine residue and a multifunctional moiety having n primary amine groups, specifically, a tris(2-aminoethyl)amine residue having two primary amine groups, is reacted with n molecules of an amine-protected iodinated amino acid compound, specifically, two molecules of tetraiodothyroformic acid,

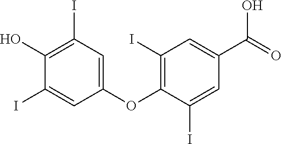 [SMILES: O=C(O)c1cc(I)c(Oc2cc(I)c(O)c(I)c2)c(I)c1]

((712c), in an amide coupling reaction where the amine groups of the multifunctional precursor compound (116a) are reacted with the carboxyl group of the tetraiodothyroformic acid (712c) to form amide linkages. The amide coupling reaction shown is performed in the presence of a carbodiimide coupling agent, specifically, EDC. The resulting compound is a protected compound in which n iodinated moieties are attached to an amine-protected peptide residue, specifically, a protected iodinated peptide compound comprising two tetraiodothyroformic acid d residues and a Boc-protected trilysine residue (not shown). The Boc-protection is then removed under acidic conditions to provide an iodinated peptide compound (714c) comprising tetraiodothyroformic acid residues and a trilysine residue. In this particular embodiment, the two tetraiodothyroformic acid residues are connected to the trilysine residue through a linkage that comprises a residue of a polyamine compound, specifically, a tris(2-aminoethyl)amine residue.

Referring now to FIG. 7D, a multifunctional precursor compound, specifically, a multifunctional precursor compound (116a) like described in FIG. 1A, which comprises a Boc-protected trilysine residue and a multifunctional moiety having n primary amine groups, specifically, a tris(2-aminoethyl)amine residue having two primary amine groups, is reacted with n molecules of an amine-protected iodinated amino acid compound, specifically, two molecules of ioxaglic acid,

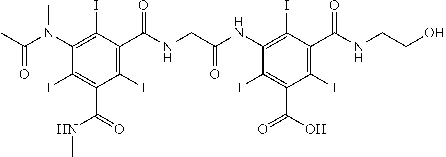 [SMILES: CNC(=O)c1c(I)c(C(=O)NCC(=O)Nc2c(I)c(C(=O)O)c(I)c(C(=O)NCCO)c2I)c(I)c(N(C)C(C)=O)c1I]

(712d), in an amide coupling reaction where the amine groups of the multifunctional precursor compound (116a) are reacted with the carboxyl group of the ioxaglic acid (712d) to form amide linkages. The amide coupling reaction shown is performed in the presence of a carbodiimide coupling agent, specifically, EDC. The resulting compound is a protected compound in which n iodinated moieties are attached to an amine-protected peptide residue, specifically, a protected iodinated peptide compound comprising two ioxaglic acid d residues and a Boc-protected trilysine residue (not shown). The Boc-protection is then removed under acidic conditions to provide an iodinated peptide compound (714d) comprising ioxaglic acid residues and a trilysine residue. In this particular embodiment, the two ioxaglic acid residues are connected to the trilysine residue through a linkage that comprises a residue of a polyamine compound, specifically, a tris(2-aminoethyl)amine residue.

Referring now to FIG. 7E, a multifunctional precursor compound, specifically, a multifunctional precursor compound (116a) like described in FIG. 1A, which comprises a Boc-protected trilysine residue and a multifunctional moiety having n primary amine groups, specifically, a tris(2-aminoethyl)amine residue having two primary amine groups, is reacted with n molecules of an amine-protected iodinated amino acid compound, specifically, two molecules of N-acetyl-thyroxine,

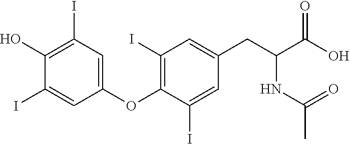 [SMILES: CC(=O)NC(Cc1cc(I)c(Oc2cc(I)c(O)c(I)c2)c(I)c1)C(=O)O]

(712e), in an amide coupling reaction where the amine groups of the multifunctional precursor compound (116a) are reacted with the carboxyl group of the N-acetyl-thyroxine (712e) to form amide linkages. The amide coupling reaction shown is performed in the presence of a carbodiimide coupling agent, specifically, EDC. The resulting compound is a protected compound in which n iodinated moieties are attached to an amine-protected peptide residue, specifically, a protected iodinated peptide compound comprising two N-acetyl-thyroxine d residues and a Boc-protected trilysine residue (not shown). The Boc-protection is then removed under acidic conditions to provide an iodinated peptide compound (714e) comprising N-acetyl-thyroxine residues and a trilysine residue. In this particular embodiment, the two N-acetyl-thyroxine residues are connected to the trilysine residue through a linkage that comprises a residue of a polyamine compound, specifically, a tris(2-aminoethyl)amine residue.

Although not shown, in embodiments where the carboxyl-functional iodinated compound has more than 1 carboxylic acid functional group, a significant excess of the iodinated species can be used during the coupling reaction with the multifunctional precursor compound to arrive at only a single carboxyl-functional iodinated compound attached to each the multifunctional precursor compound. The product can be further purified via chromatography methods or possible fractional crystallization.

In other aspects of the present disclosure, crosslinked networks are formed by reacting (a) a radiopaque peptide compound formed in accordance with the present disclosure, which comprises two or more primary-amine-containing side groups, and (b) a reactive polymer that comprises moieties that are reactive with the primary amine groups of the radiopaque peptide.

In some embodiments the crosslinked networks are hydrogels. As used herein, a “hydrogel” (also referred to as a “crosslinked hydrogel”) is a crosslinked polymer that can absorb water but does not dissolve when placed in water.

The crosslinked hydrogels of the present disclosure may be formed in vivo (e.g., using a delivery device like that described below), or such crosslinked hydrogels may be formed ex vivo and subsequently administered to a subject. The crosslinked hydrogels of the present disclosure may be used in a variety of biomedical applications, including implants, medical devices, and pharmaceutical compositions.

In various embodiments, the crosslinked hydrogel is visible under fluoroscopy. The crosslinked hydrogel may have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values).

Reactive polymers for use in the present disclosure include reactive multi-arm polymers that comprise a plurality of polymer arms linked to a core region, at least a portion of the arms comprising a hydrophilic polymer segment. One end of the hydrophilic polymer segment is covalently linked to the core region and an opposite end of the hydrophilic polymer segment is covalently linked to a reactive moiety.

In certain embodiments, at least a portion of the polymer arms comprise a hydrophilic polymer segment that has first and second ends, the first end of the hydrophilic polymer segment covalently linked to the core region, a cyclic anhydride residue having first and second ends, the first end of the cyclic anhydride residue covalently linked to the second end of the hydrophilic polymer segment, and a reactive moiety that is covalently linked to the second end of the cyclic anhydride residue.

Reactive polymers in accordance with the present disclosure include polymers having from 3 to 100 arms, for example ranging anywhere from 3 to 4 to 5 to 6 to 7 to 8 to 10 to 12 to 15 to 20 to 25 to 50 to 75 to 100 arms (in other words, having a number of arms ranging between any two of the preceding values).

Reactive moieties include moieties that comprise electrophilic groups.

Electrophilic groups may be selected, for example, from cyclic imide ester groups, such as succinimide ester groups,

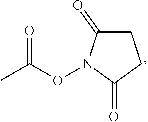

maleimide ester groups, glutarimide ester groups, diglycolimide ester groups, phthalimide ester groups, and bicyclo[2.2.1]hept-5-ene-2,3-dicarboxylic acid imide ester groups,

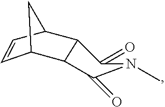

imidazole ester groups, imidazole carboxylate groups and benzotriazole ester groups, among other possibilities.

The electrophilic groups may be linked to the hydrophilic polymer segment through any suitable linking moiety, which may be selected, for example, from a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, or a linking moiety that comprises a combination of two or more of the foregoing groups, among others. In certain embodiments, the linking moiety comprises a hydrolysable ester group.

Hydrophilic polymer segments for the polymer arms can be selected from a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinylimidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain between 2 and 1000 monomer units or more, for example, ranging anywhere from 2 to 3 to 4 to 6 to 8 to 10 to 15 to 20 to 25 to 50 to 100 to 250 to 500 to 1000 monomer units (in other words range between any two of the preceding values).

In certain embodiments, the core region comprises a residue of a polyol comprising three or more hydroxyl groups, which is used to form the polymer arms. In certain beneficial embodiments, the core region comprises a residue of a polyol that contains from 3 to 100 hydroxyl groups, for example, ranging 2 to 3 to 4 to 5 to 6 to 8 to 10 to 15 to 20 to 25 to 50 to 75 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

In other embodiments, the core region comprises a silsesquioxane, which is a compound that has a cage-like silicon-oxygen core that is made up of Si—O—Si linkages and tetrahedral Si vertices. —H groups or exterior organic groups may be covalently attached to the cage-like silicon-oxygen core. In the present disclosure, the organic groups comprise polymer arms. Silsesquioxanes for use in the present disclosure include silsesquioxanes with 6 Si vertices, silsesquioxanes with 8 Si vertices, silsesquioxanes with 10 Si vertices, and silsesquioxanes with 12 Si vertices, which can act, respectively, as cores for 6-arm, 8-arm, 10-arm and 12-arm polymers. The silicon-oxygen cores are sometimes referred to as T6, T8, T10, and T12 cage-like silicon-oxygen cores, respectively (where T=the number of tetrahedral Si vertices). In all cases each Si atom is bonded to three O atoms, which in turn connect to other Si atoms. Silsesquioxanes include compounds of the chemical formula [RSiO_{3/2}]ₙ, where n is an integer of at least 6, commonly 6, 8, 10 or 12 (thereby having T₆, T₈, T₁₀ or T₁₂ cage-like silicon-oxygen core, respectively), and where R may be selected from an array of organic functional groups such as alkyl groups, aryl groups, alkoxyl groups, and polymeric arms, among others. The Ts cage-like silicon-oxygen cores are widely studied and have the formula [RSiO_{3/2}]₈, or equivalently R₈Si₈O₁₂. Such a structure is shown here:

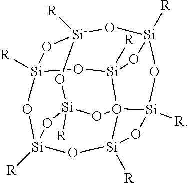

In the present disclosure, the R groups comprise the polymer arms described herein.

Reactive multi-arm polymers in accordance with the present disclosure can be formed from hydroxy-terminated precursor multi-arm polymers having arms that comprise one or more hydroxyl end groups. In some of these embodiments, the hydroxy-terminated precursor multi-arm hydrophilic polymer may be reacted with a cyclic anhydride to form an acid-end-capped precursor polymer. For example, terminal hydroxyl groups of the hydrophilic segments may be reacted with a cyclic anhydride (e.g., a glutaric anhydride compound, a succinic anhydride compound, a malonic anhydride compound, an adipic anhydride compound, a diglycolic anhydride compound, etc.) to form an acid-end-capped segment such as a glutaric-acid-end-capped segment, a succinic-acid-end-capped segment, a malonic-acid-end-capped segment, an adipic-acid-end-capped segment, a diglycolic-acid-end-capped segment, and so forth.

The preceding cyclic anhydrides, among others, may be reacted with a hydroxy-terminated precursor multi-arm hydrophilic polymer under basic conditions to form a carboxylic-acid-terminated precursor polymer comprising a carboxylic acid end group that is linked to a hydrophilic polymer segment through a hydrolysable ester group. A reactive moiety may then be linked to the carboxylic-acid-terminated precursor polymer.

In some embodiments, an electrophilic moiety may be linked to the carboxylic-acid-terminated precursor polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated precursor polymer in the presence of a suitable coupling agent (e.g., a carbodiimide coupling agent such as N,N′-dicyclohexylcarbodiimide (DCC), 1-ethyl-3-(3-dimethyl propyl) carbodiimide (EDC), N-hydroxybenzotriazole (HOBt), BOP reagent, and/or another coupling agent) to form a reactive cyclic imide ester (e.g., a succinimide ester group, a maleimide ester group, a glutarimide ester group, a phthalimide ester group, a diglycolimide ester group, a bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

In other aspects of the present disclosure, a system is provided that comprises (a) a first composition that comprises a radiopaque peptide compound as described herein and (b) a second composition that comprises a reactive polymer comprising reactive moieties as described herein, wherein the system is configured to deliver the reactive polymer and the radiopaque peptide compound under conditions such that covalent crosslinks are formed between the reactive polymer and the radiopaque peptide compound.

The first composition may be a first fluid composition comprising the radiopaque peptide compound or a first dry composition that comprises the radiopaque peptide compound, to which a suitable fluid such as water for injection, saline, etc. can be added to form a first fluid composition. In addition to the radiopaque peptide compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer, to which a suitable fluid such as water for injection, saline, etc. can be added to form a second fluid composition. In addition to the reactive polymer, the second composition may further comprise additional agents, including therapeutic agents, imaging agents, colorants, tonicity adjusting agents, suspension agents, wetting agents, and pH adjusting agents as described below.

In some embodiments, the system is configured to combine a first fluid composition comprising the radiopaque peptide compound with a second fluid comprising the reactive polymer. Upon mixing the first and second fluid compositions, the radiopaque peptide compound crosslinks with the reactive polymer, forming a crosslinked product. The first and second fluid compositions may be combined form crosslinked hydrogels, either in vivo or ex vivo.

In some embodiments, the radiopaque peptide compound is initially combined with the reactive polymer under conditions where crosslinking between the reactive polymer and the radiopaque peptide compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between the radiopaque peptide compound and the reactive polymer, thereby forming a crosslinked product.

In some embodiments, the system comprises (a) a first composition that comprises radiopaque peptide compound as described hereinabove, (b) a second composition that comprises a reactive polymer as described hereinabove, and (c) a third composition, specifically, an accelerant composition, that contains an accelerant that is configured to accelerate a crosslinking reaction between the radiopaque peptide compound and the reactive polymer.

The first composition may be a first fluid composition comprising the radiopaque peptide compound that is buffered to an acidic pH or a first dry composition that comprises the radiopaque peptide compound, to which a suitable fluid such as water for injection, saline, an acidic buffer solution, etc. can be added to form a first fluid composition comprising the radiopaque peptide compound that is buffered to an acidic pH. In some embodiments, for example, the acidic buffering composition may comprise monobasic sodium phosphate, among other possibilities. The first fluid composition comprising the radiopaque peptide compound may have a pH ranging, for example, from about 3 to about 5. In addition to the radiopaque peptide compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive polymer or a second dry composition that comprises the reactive polymer from which a fluid composition is formed, for example, by the addition of a suitable fluid such as water for injection, saline, or the first fluid composition comprising the radiopaque peptide compound that is buffered to an acidic pH. In addition to the reactive polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the radiopaque peptide compound that is buffered to an acidic pH and the second composition comprises a dry composition that comprises the reactive polymer. The first composition may then be mixed with the second composition to provide a prepared fluid composition that is buffered to an acidic pH and comprises the radiopaque peptide compound and the reactive polymer. In a particular example, a syringe may be provided that contains the first fluid composition comprising the radiopaque peptide compound that is buffered to an acidic pH, and a vial may be provided that comprises the dry composition (e.g., a powder) that comprises the reactive polymer. The syringe may then be used to inject the first fluid composition into the vial containing the reactive polymer to form a prepared fluid composition that is buffered to an acidic pH and contains the radiopaque peptide compound and the reactive polymer, which can be withdrawn back into the syringe for administration.

The accelerant composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 9 to about 11. In addition to the above, the fluid accelerant composition may further comprise additional agents, including those described below.

A prepared fluid composition that is buffered to an acidic pH and comprises the radiopaque peptide compound and the reactive polymer as described above, and a fluid accelerant composition that is buffered to basic pH as described above, may be combined form crosslinked hydrogels, either in vivo or ex vivo.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly (DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethene (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

In various embodiments, a system is provided that includes one or more delivery devices for delivering first and second compositions to a subject.

In some embodiments, the system may include a delivery device that comprises a first reservoir that contains a first fluid composition that comprises a radiopaque peptide compound as described herein and a second reservoir that contains a second fluid composition that comprises a reactive polymer as described herein, wherein the first and second fluid compositions form a crosslinked product upon mixing. In some embodiments, the system may include a delivery device that comprises a first reservoir that contains a first fluid composition that comprises the radiopaque peptide compound and the reactive polymer and is buffered to first pH, such as the prepared fluid composition previously described, and a second reservoir that contains second fluid composition, for example, a fluid accelerant composition that when combined with the first fluid composition changes a pH of the environment surrounding the radiopaque peptide compound and the reactive polymer (such as the fluid accelerant composition previously described), leading to crosslinking between the radiopaque peptide compound and the reactive polymer.

In either case, during operation, the first fluid composition and second fluid composition are dispensed from the first and second reservoirs and combined, whereupon the radiopaque peptide compound and the reactive polymer and crosslink with one another to form a crosslinked hydrogel.

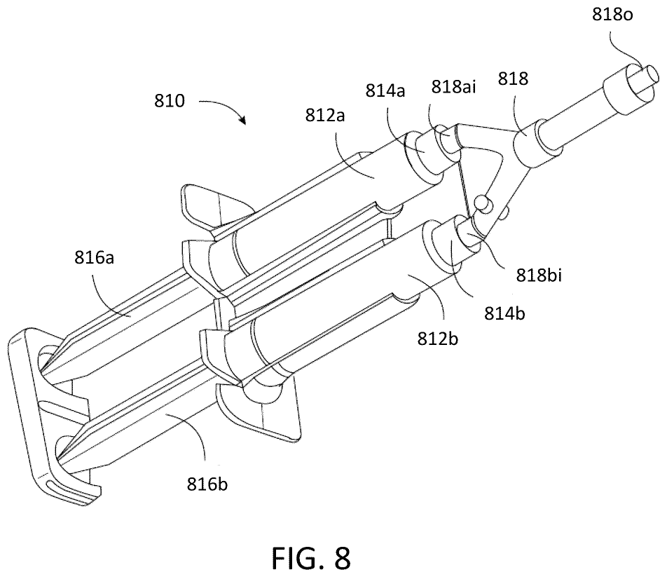

In particular embodiments, and with reference to FIG. 8, the system may include a delivery device 810 that comprises a double-barrel syringe, which includes a first barrel 812a having a first barrel outlet 814a, which first barrel contains the first composition, a first plunger 816a that is movable in the first barrel 812a, a second barrel 812b having a second barrel outlet 814b, which second barrel 812b contains the second composition, and a second plunger 816b that is movable in the second barrel 812b. In some embodiments, the device 810 may further comprise a mixing section 818 having a first mixing section inlet 818ai in fluid communication with the first barrel outlet 814a, a second mixing section inlet 818bi in fluid communication with the second barrel outlet, and a mixing section outlet 8180.

In some embodiments, the delivery device may further comprise a cannula or catheter tube that is configured to receive first and second fluid compositions from the first and second barrels. For example, a cannula or catheter tube may be configured to form a fluid connection with an outlet of a mixing section by attaching the cannula or catheter tube to an outlet of the mixing section, for example, via a suitable fluid connector such as a luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and the radiopaque peptide compound and the reactive polymer ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers, the first and second fluid compositions or a fluid admixture thereof can be injected for tissue augmentation or regeneration, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

After administration, the compositions of the present disclosure can be imaged using a suitable imaging technique. Typically, the imaging techniques is an x-ray-based imaging technique, such as computerized tomography or x-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant a therapeutic-agent-releasing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

In various embodiments, kits are provided that include one or more delivery devices for delivering the crosslinked hydrogel composition to a subject. Such systems may include one or more of the following: a syringe barrel, which may or may not contain a crosslinked hydrogel composition as described herein; a vial, which may or may not contain a crosslinked hydrogel composition as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel composition may be provided in dry form (e.g., powder form) or in a form that is ready for injection, such as an injectable hydrogel form (e.g., a suspension of crosslinked hydrogel particles).

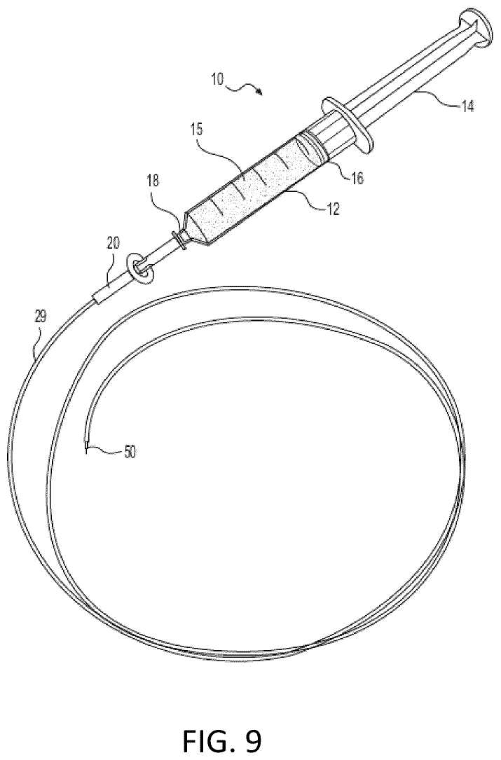

FIG. 9 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

After administration, the crosslinked hydrogel compositions of the present disclosure can be imaged using a suitable imaging technique.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intra-discal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intra-vitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A method of forming an iodinated peptide compound comprising: (a) forming a multifunctional precursor compound comprising (i) an amine-protected peptide residue that comprises two or more amino acid residues with primary-amine-containing side groups and (ii) a multifunctional moiety linked to the amine-protected peptide residue through an amide group or an ester group, the multifunctional moiety further comprising n primary amine groups, n hydroxyl groups, or n carboxyl groups, wherein n is an integer of 1 or more; (b) reacting the multifunctional precursor compound in an amide coupling reaction or an ester coupling reaction with an iodinated compound selected from an amine-functional iodinated compound that comprises an iodinated moiety, a carboxyl-functional iodinated compound that comprises an iodinated moiety, and a hydroxy-functional iodinated compound that comprises an iodinated moiety, to form an iodinated, amine-protected peptide compound comprising n iodinated moieties that are covalently attached to the amine-protected peptide residue through a residue of the multifunctional moiety; and (c) deprotecting the iodinated, amine-protected peptide compound to form the iodinated peptide compound.

2. The method of claim 1, wherein the multifunctional moiety is linked to the amine-protected peptide residue through the amide group and wherein the multifunctional moiety comprises the n primary amine groups.

3. The method of claim 2, wherein the multifunctional precursor compound is formed by a process that comprises reacting in an amide coupling reaction (i) a polyamine compound having n+1 primary amine groups with (ii) a carboxyl group at a C-terminus of an amine-protected peptide that comprises the two or more amino acid residues with the primary-amine-containing side groups.

4. The method of claim 2, wherein the n primary amine groups of the multifunctional precursor compound are reacted in an amide coupling reaction with n of the carboxyl-functional iodinated compounds to form the iodinated, amine-protected peptide compound.

5. The method of claim 1, wherein the multifunctional moiety is linked to the amine-protected peptide residue through the ester group and wherein the multifunctional moiety comprises the n hydroxyl groups.

6. The method of claim 5, wherein the multifunctional precursor compound is formed by a process that comprises reacting in an ester coupling reaction (i) a polyol compound having n+1 hydroxyl groups with (ii) a carboxyl group at a C-terminus of an amine-protected peptide that comprises the two or more amino acid residues with the primary-amine-containing side groups.

7. The method of claim 5, wherein the n hydroxyl groups of the multifunctional precursor compound are reacted in an ester coupling reaction with n carboxyl-functional iodinated compounds to form the iodinated, amine-protected peptide compound.

8. The method of claim 5, wherein the multifunctional moiety is linked to the amine-protected peptide residue through the amide group and wherein the multifunctional moiety comprises the n carboxyl groups.

9. The method of claim 8, wherein the multifunctional precursor compound is formed by a process that comprises (a) reacting in an amide coupling reaction (i) a polyamine compound having n+1 primary amine groups with (ii) a carboxyl group at a C-terminus of an amine-protected peptide that comprises the two or more amino acid residues with the primary-amine-containing side groups, the reaction product having n primary amine groups; and (b) reacting the n primary amine groups of the reaction product of step (a) with a cyclic anhydride in a ring opening reaction, thereby forming the n carboxyl groups at the sites of the n primary amine groups.

10. The method of claim 8, wherein the multifunctional precursor compound is formed by a process that comprises (a) reacting in an ester coupling reaction (i) a polyol compound having n+1 hydroxyl groups with (ii) a carboxyl group at a C-terminus of an amine-protected peptide that comprises the two or more amino acid residues with the primary-amine-containing side groups, the reaction product having n hydroxyl groups; and (b) reacting the n hydroxyl groups of the reaction product of step (a) with a cyclic anhydride in a ring opening reaction, thereby forming the n carboxyl groups at the sites of the n hydroxyl groups.

11. The method of claim 8, wherein the multifunctional precursor compound is formed by a process that comprises (a) reacting in an amide coupling reaction (i) a carboxyl-protected polycarboxylamine compound comprising at least one amine group and n protected carboxyl groups that are protected from reacting in the amide coupling reaction with (ii) a carboxyl group at a C-terminus of an amine-protected peptide that comprises the two or more amino acid residues with the primary-amine-containing side groups; and (b) deprotecting the n protected carboxyl groups, but not protected amine groups, of the reaction product of step (a).

12. The method of claim 8, wherein the multifunctional precursor compound is formed by a process that comprises (a) reacting in an ester coupling reaction (i) a carboxyl-protected hydroxycarboxylic acid compound comprising a hydroxyl group and n protected carboxyl groups that are protected from reacting in the ester coupling reaction with (ii) a carboxyl group at a C-terminus of an amine-protected peptide that comprises the two or more amino acid residues with the primary-amine-containing side groups; and (b) deprotecting the n protected carboxyl groups, but not protected amine groups, of the reaction product of step (a).

13. The method of claim 8, wherein the n carboxyl groups of the multifunctional precursor compound are reacted with n hydroxyl-functional iodinated compounds in an ester coupling reaction to form the iodinated, amine-protected peptide compound or wherein the n carboxyl groups of the multifunctional precursor compound are reacted with n amine-functional iodinated compounds in an amide coupling reaction to form the iodinated, amine-protected peptide compound.

14. The method of claim 1, wherein each of the n iodinated moieties comprise one or more iodinated aromatic groups.

15. The method of claim 14, wherein the one or more iodinated aromatic groups comprise a monocyclic or multicyclic aromatic structure that is substituted with one or more iodine groups.

16. The method of claim 15, wherein the monocyclic or multicyclic aromatic structure is further substituted with one or more hydroxyl groups and/or one or more substituents that comprise a C₁-C₄-hydroxyalkyl group.

17. The method of claim 15, wherein the monocyclic or multicyclic aromatic structure is further substituted with one or more substituents that comprise a C₁-C₄-hydroxyalkyl group and wherein the C₁-C₄-hydroxyalkyl group comprises adjacent hydroxyl groups.

18. The method of claim 1, wherein the two or more amino acids with primary-amine-containing side groups are selected from lysine, ornithine, arginine, and combinations of any two or all three of lysine, ornithine and arginine.

19. A hydrogel comprising a crosslinked reaction product of a radiopaque peptide compound and a reactive polymer that comprises moieties that react with the radiopaque peptide compound to form covalent linkages, wherein the iodinated peptide compound is provided by a method that comprises: (a) forming a multifunctional precursor compound comprising (i) an amine-protected peptide residue that comprises two or more amino acid residues with primary-amine-containing side groups and (ii) a multifunctional moiety linked to the amine-protected peptide residue through an amide group or an ester group, the multifunctional moiety further comprising n primary amine groups, n hydroxyl groups, or n carboxyl groups, wherein n is an integer of 2 or more; (b) reacting the multifunctional precursor compound in an amide coupling reaction or an ester coupling reaction with an iodinated compound selected from an amine-functional iodinated compound that comprises an iodinated moiety, a carboxyl-functional iodinated compound that comprises an iodinated moiety, and a hydroxy-functional iodinated compound that comprises an iodinated moiety, to form an iodinated, amine-protected peptide compound comprising n iodinated moieties that are covalently attached to the amine-protected peptide residue through a residue of the multifunctional moiety; and (c) deprotecting the iodinated, amine-protected peptide compound to form the iodinated peptide compound.

20. A system that comprises a first composition that comprises a radiopaque peptide compound and a second composition that comprises a reactive polymer that comprises moieties that react with the radiopaque peptide compound, wherein the system is configured to deliver the reactive polymer and the radiopaque peptide compound under conditions such that covalent crosslinks are formed between the reactive polymer and the radiopaque peptide compound, and wherein the iodinated peptide compound is provided by a method that comprises: (a) forming a multifunctional precursor compound comprising (i) an amine-protected peptide residue that comprises two or more amino acid residues with primary-amine-containing side groups and (ii) a multifunctional moiety linked to the amine-protected peptide residue through an amide group or an ester group, the multifunctional moiety further comprising n primary amine groups, n hydroxyl groups, or n carboxyl groups, wherein n is an integer of 2 or more; (b) reacting the multifunctional precursor compound in an amide coupling reaction or an ester coupling reaction with an iodinated compound selected from an amine-functional iodinated compound that comprises an iodinated moiety, a carboxyl-functional iodinated compound that comprises an iodinated moiety, and a hydroxy-functional iodinated compound that comprises an iodinated moiety, to form an iodinated, amine-protected peptide compound comprising n iodinated moieties that are covalently attached to the amine-protected peptide residue through a residue of the multifunctional moiety; and (c) deprotecting the iodinated, amine-protected peptide compound to form the iodinated peptide compound.
